(19) United States
(12) Patent Application Publication
(10) Pub. No.: US 2022/0364047 A1
(43) Pub. Date: Nov. 17, 2022

(54) MICROENCAPSULATION-BASED ISOLATION OF HUMAN PLURIPOTENT AND MULTIPOTENT STEM CELLS AND METHODS OF MAKING AND USING THE SAME

(71) Applicant: University of Maryland, College Park, College Park, MD (US)

(72) Inventors: Xiaoming He, Bethesda, MD (US); Jiangsheng Xu, Greenbelt, MD (US); James Shamul, Lanham, MD (US)

(21) Appl. No.: 17/663,372

(22) Filed: May 13, 2022

Related U.S. Application Data
(60) Provisional application No. 63/188,380, filed on May 13, 2021.

(51) Int. Cl. C12N 5/00; C12N 5/074; C12N 5/095; C12N 5/077

(52) U.S. Cl. CPC C12N 5/0012; C12N 5/0696; C12N 5/0695; C12N 5/0657; C12N 2533/80; C12N 2506/45

(57) ABSTRACT

Disclosed are microcapsule compositions and methods for encapsulating living cells. The methods include a microencapsulation approach to isolate and culture high-quality stem cells, including human iPSCs, cancer stem cells, cardiac stem cells, and the like. The microencapsulation methods are inspired by the development of blastomeres into a blastocyst within the Zona pellucida of the human female reproductive system. The bioinspired methods include encapsulation of blastomere-like cell clusters in a Zona-like microcapsule including a miniaturized hyaluronic acid-rich core and a semipermeable hydrogel shell. The cell clusters are subsequently cultured to form highly pluripotent spheroids with improved cell quality, homogeneity, and viability. Methods of use of said microcapsules are also disclosed including therapeutic uses related to human iPSC-based personalized medicines.

### CROSS-REFERENCE TO RELATED APPLICATION

This application claims the benefit of U.S. Provisional Application No. 63/188,380, filed on May 13, 2021, which is herein incorporated by reference in its entirety.

### STATEMENT REGARDING FEDERALLY SPONSORED RESEARCH

This invention was made with government support under R01EB023632B awarded by the National Institutes of Health (NIH) and under CBET1831019 awarded by the National Science Foundation (NSF). The government has certain rights in the invention.

### FIELD

This application relates generally to microcapsule compositions, methods and devices for cell microencapsulation, and methods for isolating and expanding stem cells using microcapsules.

### BACKGROUND

The creation of induced pluripotent stem cells (iPSCs) in the mid-to-late 2000s transformed the landscape of tissue engineering and regenerative medicine, due to the expanded development of personalized models and treatments for various diseases. Unlike human embryonic stem cells (ESCs), the use of human iPSCs has no ethical concerns because they are selected from somatic cells instead of cells from human embryos. Nonetheless, the promise of using human iPSCs relies heavily on maintaining a healthy, homogenous, and high-quality culture before initiating differentiation. Maintaining the pluripotency of human iPSCs under conventional 2D monolayer culture is dependent on several factors including the extent of cell dissociation, cell density for sub-culturing, cell confluency, and timing of changing medium during expansion. On the other hand, under conventional 3D suspension culture of human iPSCs, the human iPSC spheroids may form from both growth and uncontrolled fusion/merging of cell clusters from 2D culture or multiple 3D spheroids. This may result in highly heterogeneous human iPSC spheroids in terms of both size and quality/pluripotency. Therefore, heterogeneity resulting from spontaneous differentiation and uncontrolled fusion under conventional 2D and 3D cultures is a headache for human iPSC research and applications.

Several methods have been reported to address these issues including the development of a defined culture medium with specific supplements/chemicals to enhance/preserve pluripotency-promoting signaling pathways and/or inhibit signaling pathways associated with differentiation, fluorescence and magnetic-activated cell sorting, microwell culture, and genome engineering. However, the fold changes of human pluripotent stem cell (hPSC) markers (e.g., NANOG, SOX2, OCT4, and SSEA-4) are not robust and/or not all quantified for the previous methods to obtain high-quality human PSCs. Moreover, there are issues or concerns with these methods including scalability, high cost, and/or low cell viability due to their impact on metabolism and nutrient transport. A potential strategy for addressing the issues is to isolate and purify high-quality human iPSCs from a heterogeneous culture. Due to the aforementioned spontaneous differentiation, only a subset of human iPSCs may have high pluripotency in conventional 2D and 3D cultures. Traditionally, well-experienced researchers could manually remove differentiated cells by using a fine glass/plastic tip under a microscope. In addition, efforts have been made to develop technologies for removing differentiated cells. However, these methods may be highly labor-intensive, lack precision, and/or rely heavily on personal experience and skills.

Recently, several studies have reported using core-shell or droplet microfluidics for encapsulation and culture of stem cells including mesenchymal stem cells and murine pluripotent stem cells, and such culture methods have been shown to improve the pluripotency of murine pluripotent stem cells. However, no such study has been reported for human iPSCs (as with human ESCs) although a recent study showed core-shell hydrogel encapsulation could protect human iPSCs from damage by shear stress during dynamic culture in a bioreactor. This is probably because human iPSCs are notoriously much more difficult to culture/handle than all other types of human and murine stem cells. Surprisingly, no approaches have been reported to culture human iPSCs by mimicking strategies used by nature to culture and generate pluripotent ESCs during early embryonic development in humans (and other mammals). In nature, a semipermeable hydrogel shell known as the zona pellucida (Zona) is used to semi-enclose a small cluster/aggregate of blastomeres in a hydrogel core of ˜100-300 μm in diameter. The blastomeres further develop into a blastocyst containing a highly pluripotent inner cell mass (ICM) in ˜4 days (for humans) in the female reproductive system, where HA is a physiologically ubiquitous glycosaminoglycan and plays an important role during early embryonic development.

One application of core-shell encapsulation methods is the isolation and proliferation of CSCs. CSC theory is based on the presence of a sub-population of tumorigenic stem-like cells with true multipotency and asymmetric division ability, which enable these cells to self-renew, differentiate into specialized cell types and develop into cancer. Indeed, the phenotype of “cancer stemness” may be the driving force behind carcinogenesis, and CSCs may contribute to chemo- or radio-resistance and metastasis. Increasing evidence shows that CSCs are not only present in leukemia but also in various solid tumors, including lung cancer. Although lung CSCs can be isolated from side populations through specific markers such as CD133 and aldehyde dehydrogenase, it remains difficult to maintain the stemness characteristics of CSCs in vitro for detailed studies. Core-shell encapsulation methods may therefore provide an opportunity to both physically isolate individual CSCs and to maintain the phenotype of cancer stemness (e.g., maintaining a viable, homogenous CSC clone population), thereby enabling improved study and targeting of CSCs.

### SUMMARY

The disclosure provides microcapsule compositions, methods for microcapsule formation, and methods for isolating and expanding stem cells using microcapsules. The bioinspired methods include encapsulation of cells and cell clusters in a Zona-like microcapsule.

In some embodiments, the microcapsule composition for isolating and expanding cells includes a cell or cell cluster suspended in a core hydrogel/solution. The core hydrogel/solution is rich in hyaluronic acid (HA). Also disclosed are methods of encapsulating cells including suspending cells and cell clusters in a microcapsule with average diameter of less than about 1,000 microns.

Further disclosed is a method for isolating and expanding human iPSC or human IPSC clusters including encapsulating human iPSC clusters in a core, wherein the core has a volume ranging from about 0.005 to 500 nanoliters. In some embodiments, the human iPSC or human IPSC cluster may release autocrines or paracrines inside of the microcapsule.

In embodiments, stem cells may be encapsulated and proliferated within the core of the microcapsule. The core contains a core hydrogel/solution that may include hyaluronic acid, sodium alginate, collagen, carboxymethyl cellulose, proteins, cells, and other biomolecules. The stem cells may include organ-specific stem cells, multipotent stem cells, cardiac stem cells, cancer stem cells, glioblastoma stem cells, and the like. An isotonic solution may be applied to the microcapsule to release the expanded cells for further differentiation using standard 2D methods.

### BRIEF DESCRIPTION OF THE DRAWINGS

For a detailed description of the preferred embodiments of the present disclosure, reference will now be made to the accompanying drawings wherein:

FIG. 1A-1C provide a diagrammatic approach for isolation and culture of high-quality human iPSCs. FIG. 1A shows a schematic illustration of the proliferation of blastomeres into pluripotent inner cell mass (ICM) in zona pellucida (Zona) during early embryonic development. FIG. 1B shows bioinspired microencapsulation for isolating high pluripotent iPSCs. FIG. 1C shows that, after ˜4 days of proliferation, the highly pluripotent individual human iPSC clusters encapsulated in the Zona-like microcapsules grow into “ICM-like” 3D human iPSC spheroids in each microcapsule.

FIG. 2A-2C depict a strategy that can be used to isolate and culture highly pluripotent human iPSCs among a conventionally cultured heterogeneous human iPSC population. FIG. 2A is a diagram showing the inlets and microchannel system in the non-planar microfluidic device. FIG. 2B shows real-time images showing a human iPSC cluster in the core solution surrounded by a shell (i.e., sodium alginate) solution at the flow focusing junction of the non-planar device. The dark arrows indicate the locations of the human iPSC cluster in the core flow, and the white arrows indicate the interface of the core and the shell flows. FIG. 2C is a diagram with a schematic illustration of the formation of a human iPSC cluster-laden core-shell microcapsule (“microcapsule”) at the flow focusing junction.

FIG. 3A-3B show that pluripotent human iPSC spheroids may be cultured in 2D while maintaining high pluripotency. FIG. 3A is an image of iPSC clusters. FIG. 3B is an image of a Zona-like hydrogel core-hydrogel shell microcapsule. FIG. 3C is an image of the final iPSC cluster-laden microcapsule product.

FIG. 4A is a histogram showing the distribution of the number of iPSCs in each cluster. FIG. 4B shows core sizes and total sizes (in diameter) of the microcapsules together with a schematic illustration of a human iPSC cluster encapsulated in a Zona-like core-shell microcapsule. FIG. 4C is an illustration of the microcapsule including the shell, iPSC cluster, and HA-rich core.

FIG. 5A-5E show representative images of the microcapsule at various flow rates including 50 microliters per hour (FIG. 5A), 100 microliters per hour (FIG. 5B), 150 microliters per hour (FIG. 5C), 200 microliters per hour (FIG. 5D), and 250 microliters per hour (FIG. 5E). FIG. 5F shows the tunable core size of the microcapsules at indicated core flow rates.

FIG. 6A is a schematic illustration of the microencapsulation strategy for isolating and culturing highly pluripotent human iPSCs. FIG. 6B are representative phase contrast images showing the morphology of human iPSC clusters in the core of microcapsules. FIG. 6C-6F show phase contrast images and fluorescence images including a phase contrast image before encapsulation (FIG. 6C), a phase contrast image after encapsulation (FIG. 6E), a fluorescence image before encapsulation (FIG. 6D), and a fluorescence image after encapsulation (FIG. 6F).

FIG. 7A shows the quantitative analysis associated with FIGS. 6D and 6F tracking cell viability before and after encapsulation.

FIG. 8A-8E show representative phase contrast images including the proliferation of human iPSC clusters over 4 days in the core of the HA-rich core-shell microcapsules. The images include shell conditions of 2% alginate plus core conditions of 0.5% HA+1% alginate (FIG. 8A), shell conditions of 0.5% HA+1% alginate with equal core conditions (FIG. 8B), shell conditions of 0.5% HA+2% alginate with equal core conditions (FIG. 8C), shell conditions at 2% alginate plus core conditions of 1% alginate (FIG. 8D), and shell conditions at 2% alginate plus core at 0.5% CMC+1% alginate (FIG. 8E).

FIG. 9A-9C are schematic illustrations and representative phase contrast images showing the formation of human iPSC spheroids under conventional suspension culture, in which aggregation/fusion of human iPSC clusters/spheroids may occur. FIG. 9A is an illustration depicting aggregation/fusion of human iPSC clusters/spheroids. FIG. 9B is a phase contrast image at time zero while FIG. 9C is a phase contrast image at time 18 hours.

FIG. 10A-10E show time-sequence images of two smaller human iPSC spheroids fusing into a larger spheroid under the conventional suspension culture at time zero (FIG. 10A), 20 minutes (FIG. 10B), 45 minutes (FIG. 10C), 70 minutes (FIG. 10D), and 130 minutes (FIG. 10E).

FIG. 11A shows the size change of human iPSC spheroids over time under both the conventional 3D suspension (3DSusp) culture and microencapsulation (3DEncap) culture. The inset images of live/dead (green/red) staining show that both culture methods can form highly viable human iPSC spheroids. FIG. 11B shows the size distributions of human iPSC spheroids obtained from the conventional 3D suspension culture and bioinspired 3D microencapsulation culture.

FIG. 12A shows flow cytometry peaks for NANOG (FIG. 12A), OCT4 (FIG. 12B), SOX2 (FIG. 12C), and SSEA-4 (FIG. 12D) in human iPSCs under bioinspired 3D microencapsulation culture (“P”) versus conventional 2D and 3D suspension cultures (“0” and “B”, respectively).

FIG. 13A-13D shows the quantitative analysis corresponding to the data provided in FIG. 12A-12D, including analysis for NANOG (FIG. 13A), OCT4 (FIG. 13B), SOX2 (FIG. 13C), and SSEA-4 (FIG. 13D). For the quantitative analysis, the peak intensity for the two 3D culture conditions is normalized to that for the 2D culture. Data are presented as mean±standard deviation (s.d.). One-way analysis of variance (ANOVA) with Tukey's post hoc analysis was used to assess statistical significance, *p<0.05, **p<0.01. Taken together, FIG. 12A-12D show that the microcapsules selectively isolate stem cells having reduced differentiation resistance relative to conventional 2D and 3D methods, as indicated by a higher expression of pluripotency markers.

FIG. 14A shows the cumulative percentage of beating spheroids showing a significantly higher percentage of beating spheroids from 3D_{Encap} culture than that of 3D_{Susp} culture.

FIG. 14B-14C shows flow cytometry analysis of the cells after 10 days of guided cardiac differentiation showing a significantly higher percentage of cardiac troponin T (cTnT, a protein marker specific to cardiomyocytes) positive cells in 3D_{Encap} group than that in 3D_{Susp} group (FIG. 14B). Cells from human iPSC spheroids before differentiation are used as the control (FIG. 14C). Data are presented as mean±standard deviation (s.d.). Student's t-test (unpaired, two-tailed) was used to assess statistical significance.

FIG. 15 shows a schematic illustration showing conversion of 3D human iPSC spheroids from 3D_{Susp} and 3D_{Encap} cultures to 2D (3D_{Susp}-2D and 3D_{Encap}-2D) human iPSCs on Matrigel-coated plates. The human iPSCs attach on the substrate within 24 h for both conditions. Scale bar: 100 μm.

FIG. 16A-16C show the phase contrast images corresponding to FIG. 15 including timepoints at time zero (FIG. 16A), 8 hours (FIG. 16B), and 24 hours (FIG. 16C).

FIG. 17A-17D depict flow cytometry peaks for expression of pluripotency markers NANOG (FIG. 17A), OCT4 (FIG. 17B), SOX2 (FIG. 17C), and SSEA-4 (FIG. 17D) in the 2D attached 3D_{Susp}-2D and 3D_{Encap}-2D human iPSCs converted from the 3D_{Susp} and 3D_{Encap} human iPSC spheroids, respectively. Data are presented as mean±standard deviation (s.d.). One-way analysis of variance (ANOVA) with Tukey's post hoc analysis was used to assess statistical significance, *p<0.05, **p<0.01.

FIG. 18A-18D is the quantitative analysis corresponding to FIG. 17A-17D for expression of pluripotency markers NANOG (FIG. 18A), SOX2 (FIG. 18B), OCT4 (FIG. 18C), and SSEA-4 (FIG. 18D) in the 2D attached 3D_{Susp}-2D and 3D_{Encap}-2D human iPSCs converted from the 3D_{Susp} and 3D_{Encap} human iPSC spheroids, respectively. Data are presented as mean±standard deviation (s.d.). One-way analysis of variance (ANOVA) with Tukey's post hoc analysis was used to assess statistical significance, *p<0.05, **p<0.01.

FIG. 19A-19C are phase contrast images showing growth of circulating tumor cells (“CTCs”) into a cell colony at day 1 (FIG. 19A), day 12 (FIG. 19B), and day 21 (FIG. 19C). Notably, the bioinspired encapsulation strategy is capable of isolating cancer stem cells from circulating tumor cells (CTCs).

FIG. 20A-20C are phase contrast images showing growth of circulating glioblastoma (GBM) cells isolated from glioblastoma tumors, showing isolation of true glioblastoma stem cells (GSCs). The phase contrast images show growth at day 1 (FIG. 20A), day 12 (FIG. 20B), day 21 (FIG. 20C).

### DETAILED DESCRIPTION

Disclosed are methods and devices for encapsulating living cells, as well as methods of using said microencapsulated cells for 3D cell culture, 2D cell culture, differentiation, tissue engineering, and the like.

### Definitions

So that aspects of the present disclosure may be more readily understood, certain terms are first defined. Unless defined otherwise, all technical and scientific terms used herein have the same meaning as commonly understood by one skilled in the art to which embodiments of the present disclosure pertain. Many methods and materials similar, modified, or equivalent to those described herein can be used in the practice of the embodiments of the present disclosure without undue experimentation. In describing and claiming the embodiments of the present disclosure, the following terminology will be used in accordance with the definitions set out below.

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the context clearly indicates otherwise. Similarly, the word “or” is intended to include “and” unless the context clearly indicates otherwise. The word “or” means any one member of a particular list and also includes any combination of members of that list. Further, all units, prefixes, and symbols may be denoted in their SI accepted form.

Numeric ranges recited within the specification, including ranges of “greater than,” “at least”, or “less than” a numeric value, are inclusive of the numbers defining the range and include each integer within the defined range. For example, when a range of “1 to 5” is recited, the recited range should be construed as including ranges “1 to 4”, “1 to 3”, “1-2”, “1-2 & 4-5”, “1-3 & 5”, and the like.

“About” refers to variation in the numerical quantity that can occur, for example, through typical measuring techniques and equipment, with respect to any quantifiable variable, including, but not limited to, mass, volume, time, and temperature. Further, given solid and liquid handling procedures used in the real world, there is certain inadvertent error and variation that is likely through differences in the manufacture or purity of the ingredients used to make the compositions or carry out the methods and the like. The term “about” also encompasses these variations. Whether or not modified by the term “about,” the claims include equivalents to the quantities.

“Alginate” is a collective term used to refer to linear polysaccharides formed from β-D-mannuronate and β-L-guluronate in any M/G ratio, as well as salts and derivatives thereof.

“Biocompatible” generally refers to material and any metabolites or degradation products thereof that are non-toxic and do not cause any significant adverse effects to biological cells and/or mammalian subjects.

“Bioactive agent” generally refers to substances that can influence an organism, tissue, or cell. Examples include biomolecules, enzymes, drugs, vitamins, phytochemicals, and bioactive compounds. Bioactive agents can be incorporated into polymers, which have applications in drug delivery and commercial production of household goods and biomedical devices. In drug delivery systems, bioactive agents are loaded into enzyme-responsive polymers which can then be cleaved by target enzymes. Activation of the bioactive agents leads to the release of therapeutic cargos.

“Biomolecule” generally refers to molecules present in organisms that influence biological processes, such as cell division, morphogenesis, or development. Biomolecules include large macromolecules (or polyanions) such as proteins, carbohydrates, lipids, and nucleic acids, as well as small molecules such as primary metabolites, secondary metabolites, and natural products. A more general name for this class of material is biological materials. In the present context, autocrine is an example of a biomolecule that influences stem cell proliferation and health.

“Cardiac Stem Cells” are multipotent cells residing in the adult mammalian heart that are capable of self-renewing and generating coronary vessels and heart muscle cells called cardiomyocytes. Cardiac stem cells can contribute to new cardiomyocyte formation following experimental myocardial infarction in mice. Human iPSC spheroids may be differentiated into cardiac stem cells by washing microcapsules with phosphate-buffered saline (PBS), then applying cardiac differentiation mediums A and B as described in the Examples. Notably microcapsules may be washed in phosphate-buffered saline (PBS), sodium citrate, EDTA, or another ion chelation solution known in the art. Cardiac stem cells are clonogenic, self-renewing, and multipotent in vitro and in vivo, capable of generating the three major cell types of the myocardium: myocytes, smooth muscle, and endothelial vascular cells. They express several markers of stemness (i.e., Oct3/4, Bmi-1, NANOG) and have significant regenerative potential in vivo. When cloned in suspension they form cardiospheres, which when cultured in a myogenic differentiation medium, differentiate into beating cardiomyocytes.

“Cancer Stem Cells” (CSCs) are cancer cells (found within tumors or hematological cancers) that possess characteristics associated with normal stem cells, specifically the ability to give rise to all cell types found in a particular cancer sample. CSCs are therefore tumorigenic (tumor-forming), in contrast to other non-tumorigenic cancer cells. CSCs may generate tumors through the stem cell processes of self-renewal and differentiation into multiple cell types. Such cells persist in tumors as a distinct population and cause relapse and metastasis by giving rise to new tumors. CSCs can be distinguished from other cells within the tumor by symmetry of their cell division and alterations in their gene expression. A number of cell surface markers such as CD44, CD24, and CD133 are often used to identify and enrich CSCs. A regulatory network consisting of microRNAs and Wnt/β-catenin, Notch, and Hedgehog signaling pathways controls the CSC properties.

“Cancer” refers to a class of diseases or conditions in which abnormal cells divide without control and can invade nearby tissues. A malignant cancer is one in which a group of tumor cells displays one or more of uncontrolled growth (e.g., division beyond normal limits), invasion (e.g., intrusion on and destruction of adjacent tissues), and metastasis (e.g., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancers form tumors, but some, e.g., leukemia, and some blood cancers, do not necessarily form tumors. For those cancers that form tumors, the terms cancer (cell) and tumor (cell) are used interchangeably. The amount of a tumor in an individual is the “tumor burden” which can be measured as the number, volume, or weight of the tumor.

“Cell” refers to any living cell. The cell may be xenogeneic, autologous, or allogeneic. The cell can be a primary cell obtained directly from a plant or animal, such as a mammal. The cell may also be a cell derived from the culture and expansion of a cell obtained from a plant or animal. For example, the cell may be a stem cell. Immortalized cells are also included within this definition. In some embodiments, the cell has been genetically engineered to express a recombinant protein and/or nucleic acid.

“Cluster” refers to a three-dimensional formation of cells that arranges itself during proliferation into a small grouping of cells (e.g., 2-15 cells). A cluster can eventually grow into a spheroid of cells.

“Culture media” and “culture medium” are used interchangeably and refer to a solid or a liquid substance used to support the growth of cells (e.g., stem cells). Preferably, the culture media as used herein refers to a liquid substance capable of maintaining stem cells in an undifferentiated state. The culture media can be a water-based media which includes a combination of ingredients such as salts, nutrients, minerals, vitamins, amino acids, nucleic acids, proteins such as cytokines, growth factors and hormones, all of which are needed for cell proliferation and are capable of maintaining stem cells in an undifferentiated state. For example, a culture media can be a synthetic culture media supplemented with the necessary additives as is further described herein. In some embodiments, the cell culture media can be a mixture of culture media. Preferably, all ingredients included in the culture media of the present disclosure are substantially pure and tissue culture grade.

“Culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” means any proliferation or division of cells.

“Differentiation” describes the process whereby an unspecialized cell acquires the features of a specialized cell such as a heart, liver, or muscle cell. As used herein, the term “differentiates or differentiated” defines a cell that takes on a more committed (“differentiated”) position within the lineage of a cell. As used herein, the term “differentiates or differentiated” defines a cell that takes on a more committed (“differentiated”) position within the lineage of a cell.

“Embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell.

“Hyaluronic acid” (HA) is a glycosaminoglycan present in many tissues throughout the body that plays an important role in embryonic development, wound healing, and angiogenesis. HA is an anionic, nonsulfated glycosaminoglycan distributed widely throughout connective, epithelial, and neural tissues. It is unique among glycosaminoglycans as it is non-sulfated, forms in the plasma membrane instead of the Golgi apparatus and can be very large in size.

“Hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer that forms a gel which is not toxic to living cells and allows sufficient diffusion of oxygen and nutrients to the entrapped cells to maintain viability.

“Induced pluripotent stem cells” (iPSCs) refers to immature cells that have been derived from reprogrammed adult somatic cells and have regained their capacity to differentiate into any type of cell found in the body. The reprogrammed cells are functionally and morphologically analogous to embryonic stem cells (ESCs) and can differentiate into all three germ layers. iPSCs can be reprogrammed from a multitude of somatic and stem cells. They have large self-renewal capability and can be used to derive patient disease-specific tissues and for drug screening. Due to the high differentiation potential, iPSCs are a promising cell model to promote cell differentiation for the regeneration of all somatic cell types (e.g., craniofacial tissues, such as osteoblasts, odontoblasts, and others).

“Isotonic solution” refers to PBS buffer and/or other isotonic solutions used to liquefy the alginate hydrogel in order to release iPSC spheroids from the microcapsules. A solution is isotonic when its effective osmole concentration is the same as that of another solution. In biology, the solutions on either side of a cell membrane are isotonic if the concentration of solutes outside the cell is equal to the concentration of solutes inside the cell. In this case the cell neither swells nor shrinks because there is no concentration gradient to induce the diffusion of large amounts of water across the cell membrane. Water molecules freely diffuse through the plasma membrane in both directions, and as the rate of water diffusion is the same in each direction, the cell will neither gain nor lose water.

“Microcapsule” refers to a particle or capsule having a mean diameter of about 50 μm to about 1,000 μm, formed of a cross-linked hydrogel shell surrounding a biocompatible core hydrogel/solution. The microcapsule may have any shape suitable for cell encapsulation. The microcapsule may contain one or more cells dispersed in the biocompatible core hydrogel/solution, cross-linked hydrogel, or combination thereof, thereby “encapsulating” the cells. The core hydrogel/solution can include hyaluronic acid, alginate, proteins, cells, bioactive agents, and other biomolecules.

“Microfluidic Device” refers to a device that includes one or more microfluidic channels, one or more microfluidic valves, one or more microfluidic chambers, or combinations thereof, and are configured to carry, store, transport, combine, and/or react component solutions in fluid volumes of less than ten milliliters (e.g., in fluid volumes of 5 mL or less, in fluid volumes of 2.5 mL or less, or in fluid volumes of 1.0 mL or less) to form microcapsules.

“Microfluidic Channel” refers to a feature within a microfluidic device that forms a path, such as a conduit, through which one or more fluids can flow. In some embodiments, microfluidic channels have at least one cross-sectional dimension that is in the range from about 10 microns to about 750 microns.

“Spheroids” refer to a three-dimensional cell culture that arranges itself during proliferation into sphere-like formations. Unlike two-dimensional, monolayer cell cultures, in which cells interact with the substrate they grow on, 3D cell cultures can grow into spheroids that promote cell-to-cell connectivity. Under conventional 3D suspension culture of iPSCs (e.g., human iPSCs), the human iPSC spheroids may form from both growth and uncontrolled fusion/merging of cell clusters from 2D culture or multiple 3D spheroids. This may result in heterogenous spheroids in terms of both size and quality/pluripotency. The microcapsule compositions disclosed herein only allow said spheroids to form from growth and not uncontrolled fusion/merging of cell clusters, thus resulting in uniform size distribution, improved quality, and high pluripotency.

“Stem cell” refers to a cell that is in an undifferentiated or partially differentiated state and has the capacity for self-renewal and/or to generate differentiated progeny. Self-renewal is defined as the capability of a stem cell to proliferate and give rise to more stem cells, while maintaining its developmental potential (i.e., totipotent, pluripotent, multipotent, etc.). The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural stem cells (NSCs). In some embodiments, the stem cells or progenitor cells can be embryonic stem cells.

The below disclosure provides next-generation microcapsules, microencapsulation methods, and new uses of said microcapsules. The herein disclosed methods of encapsulation may be used with human iPSCs, ESCs, multipotent CSCs, multipotent tissue-specific stem cells, and organ-specific stem cells. The benefits of the disclosed microcapsules are numerous, including the ability to selectively isolate cells with reduced differentiation resistance. This allows for the culture and expansion of uniquely homogenous, high quality, and highly pluripotent cells. In contrast to conventional 2D and 3D expansion methods, the disclosed microcapsules only allow for cell proliferation from the growth of the encapsulated cells (and not from fusion/merging events of cells and clusters). Using conventional techniques, fusion/merging of cells and clusters results in the development of highly heterogenous spheroids (e.g., genetically and morphologically heterogenous), low cell viability, low pluripotency, and the like. Finally, the microcapsule compositions disclosed herein represent a major advance in the field, including use of an HA-rich core and other unique materials integrated into the shell, shell hydrogel, and core hydrogel/solution.

Microcapsules

Disclosed are microcapsules containing a core and a shell. As described above, methods for isolating and expanding cells, including human iPSCs or human iPSC clusters, are provided. The herein disclosed methods for isolating and expanding human iPSCs and/or human iPSC clusters include the steps of encapsulating human iPSC clusters in an alginate hydrogel core adapted to integrate bioactive agents, UV-active compounds, a variety of cell types, and various other elements. The iPSC clusters may proliferate into highly pluripotent iPSC spheroids after 3 to 5 days of miniaturized culture. In other embodiments, said clusters proliferate within 2 to 6 days, 1 to 7 days, or about 4 days. In embodiments, the highly pluripotent iPSC spheroids may optionally be released from the microcapsules by liquifying the alginate hydrogel using a liquifying solution such as an isotonic solution (e.g., PBS, sodium citrate, and the like). In embodiments, said released highly pluripotent iPSC spheroids may be further cultured in 2D while maintaining high pluripotency. In one embodiment, the cell culture media includes StemFlex medium and Rock inhibitor, in addition to several other elements as described below.

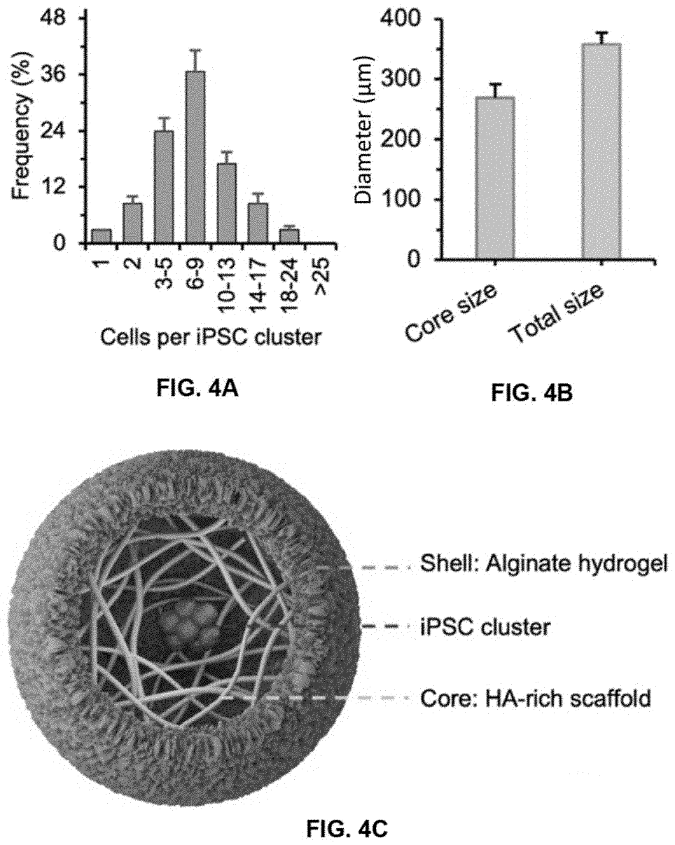

As shown in FIG. 4C, in embodiments the microcapsule includes a shell, iPSC cluster, and HA-rich core. In embodiments, microcapsules are disclosed including a core comprising hyaluronic acid, sodium alginate, and various other materials. Further, in embodiments a shell enclosing the core is produced via a mild process that ensures improved differentiation, improved isolation, homogeneity, high efficiency, and immediate cell viability. In some embodiments, the microcapsules include a core ranging from about 0.005 to 500 nanoliters in volume. The core encapsulates a stem cell or stem cell cluster (e.g., a single iPSC or human iPSC cluster) suspended in a core solution and/or scaffold. The core solution may include hyaluronic acid (HA), sodium alginate, and other compounds. The microcapsules may have an average diameter ranging from about 30 microns to about 1,000 microns. Notably, a diameter of 20-1,000 micron corresponds to volume of about 0.005-500 nanoliters.

The stem cell or stem cell cluster encapsulated in the core may include pluripotent stem cells, multipotent stem cells, cardiac stem cells, cancer stem cells, and the like. The core can contain a variety of cells including bacterial cells, mammalian cells, cell clusters, and the like suspended or encapsulated in a core hydrogel/solution. In one example, the core hydrogel/solution can be a viscous aqueous liquid or a hydrogel. In some embodiments, the core hydrogel/solution contains proteins suitable for promoting a cell activity, such as survival, attachment, growth, pluripotency, or differentiation. In some embodiments, the protein of the core hydrogel/solution can be collagen, fibrin, gelatin, elastin, or elastin-like polypeptides (ELPs), or a derivative thereof. In some embodiments, the core hydrogel/solution comprises carboxymethyl cellulose (CMC) and/or collagen. In contrast to previous systems, the core and core hydrogel/solution may comprise a heterogenous mixture of solutions, proteins, and the like.

In embodiments, the shell has a thickness ranging from about 10 microns to about 100 microns. The shell surrounding the core also includes a semipermeable alginate hydrogel, wherein the core hydrogel/solution and alginate hydrogel are distinct in their chemical composition. In embodiments, the shell is permeable to micronutrients and modestly permeable to HA. The expansion of stem cells in fact is optimized to correspond with the slow diffusion of HA across and out of the alginate hydrogel shell over the course of about 4-7 days. The shell surrounding the core can also include a biocompatible hydrogel (e.g., a “shell hydrogel”). One advantage of the disclosed microcapsules is the ability of the core hydrogel/solution and the biocompatible hydrogel to be distinct in their chemical composition or concentration. For example, the core hydrogel/solution can be an aqueous liquid while the shell can be a hydrogel surrounding the liquid core. Furthermore, the core may be comprised of heterogenous solutions, proteins, and other elements. In embodiments, the shell comprises a hydrogel (e.g., also referred to herein as “shell hydrogel”) that may include the above-mentioned hydrogel components.

In some embodiments, the core and the shell are formed so as to have distinct physical properties. For example, the core and the shell of the microcapsules can be fabricated to each have a different modulus of elasticity, density, polarity, thickness, hydrophobicity/hydrophilicity, or combinations thereof.

Examples of materials which can be used to form a suitable hydrogel (e.g., in the core, shell, or combinations thereof) include polysaccharides such as alginate, polyphosphazenes, poly(acrylic acids), poly(methacrylic acids), poly(alkylene oxides), poly(vinyl acetate), poly(acrylamides) such as poly(N-isopropylacrylamide), polyvinylpyrrolidone (PVP), and copolymers and blends of each. In some embodiments, block copolymers can be used. For example, poloxamers containing a hydrophobic poly(alkylene oxide) segment (i.e., polypropylene oxide) and hydrophilic poly(alkylene oxide) segment (i.e., polyethylene oxide) can be used.

In general, the polymers used to form the core and shell are at least partially soluble in aqueous solutions, such as water, buffered salt solutions, or aqueous alcohol solutions. In some embodiments, the polymers have polar groups, charged groups, acidic groups or salts thereof, basic groups or salts thereof, or combinations thereof. Examples of polymers with acidic groups include poly(phosphazenes), poly(acrylic acids), poly(methacrylic acids), poly(vinyl acetate), and sulfonated polymers, such as sulfonated polystyrene. Copolymers having acidic side groups formed by reaction of acrylic or methacrylic acid and vinyl ether monomers or polymers can also be used. Examples of acidic groups include carboxylic acid groups and sulfonic acid groups.

Examples of polymers with basic groups include poly(vinyl amines), poly(vinyl pyridine), poly(vinyl imidazole), and some imino substituted polyphosphazenes. Nitrogen-containing groups in these polymers can be converted to ammonium or quaternary salts Ammonium or quaternary salts can also be formed from the backbone nitrogens or pendant imino groups. Examples of basic groups include amino and imino groups.

In certain embodiments, the biocompatible hydrogel-forming polymer is a water-soluble gelling agent. In certain embodiments, the water-soluble gelling agent is a polysaccharide gum, such as a polyanionic polysaccharide. In some cases, cells or cell clusters are encapsulated using an anionic polymer such as alginate to form a microcapsule shell, core, or combinations thereof.

In some embodiments, the core hydrogel/solution comprises a viscous aqueous solution. For example, in some embodiments, the core hydrogel/solution can have a viscosity that is at least two times, five times, six times, eight times, ten times, or twenty times the viscosity of water at 25° C. In certain embodiments, the core solution can have a viscosity that is at least two times, five times, six times, eight times, ten times, or twenty times the viscosity of ethylene glycol at 25° C.

In some embodiments, cells or cell clusters may be encapsulated via an anionic polymer to form a hydrogel core solution (e.g., core). The hydrogel core solution can optionally be crosslinked, if desired. Said crosslinking may be UV activated and/or facilitated by the integration of UV-active compounds and/or cells into the shell. The core hydrogel/solution (e.g., the core) can be formed from viscous solutions, such as solutions of cellulose and its derivatives (e.g., carboxymethyl cellulose).

In embodiments, a variety of compounds have adapted for cell encapsulation. For example, mammalian and non-mammalian polysaccharides may be used for cell encapsulation. These materials can be used, alone or in part, to form the core, the shell, or both the core and the shell. Exemplary polysaccharides adapted for use in a core/hydrogel solution include alginate, chitosan, glycosaminoglycans (e.g., hyaluronic acid), and/or chondroitin sulfate. Further examples of glycosaminoglycans include chondroitin sulfate, keratan sulfate, heparin, dermatan sulfate, hyaluronate and heparan sulfate. Heparan sulfates are preferred embodiments of the present invention. As used herein, the term glycosaminoglycan also extends to encompass those molecules that are glycosaminoglycan conjugates. An example of a glycosaminoglycan conjugate is a proteoglycosaminoglycan (proteoglycan) wherein a peptidic component is covalently bound to an oligosaccharide component. In embodiments, alginate and chitosan form crosslinked hydrogels under certain solution conditions, while HA and chondroitin sulfate are preferably modified to contain crosslinkable groups to form a hydrogel. In embodiments, a variety of polysaccharides may be adapted for use in the core/hydrogel solution including any long chain polymeric carbohydrate composed of monosaccharide units bound together by glycosidic linkages.

In some embodiments, the core, the shell, or both the core and the shell comprise alginate or derivative thereof. Alginates are a family of unbranched anionic polysaccharides derived primarily from brown algae which occur extracellularly and intracellularly at approximately 20% to 40% of the dry weight. The 1,4-linked α-1-guluronate (G) and β-D-mannuronate (M) are arranged in homopolymeric (GGG blocks and MMM blocks) or heteropolymeric block structures (MGM blocks). Cell walls of brown algae also contain 5% to 20% of fucoidan, a branched polysaccharide sulfate ester with 1-fucose the-sulfate blocks as the major component. Commercial alginates are often extracted from algae washed ashore, and their properties depend on the harvesting and extraction processes. Although the properties of the hydrogel can be controlled to some degree through changes in the alginate precursor (molecular weight, composition, and macromer concentration), alginate does not degrade, but rather dissolves when the divalent cations are replaced by monovalent ions.

Alginate can form a gel in the presence of divalent cations via ionic crosslinking. This is induced by the addition of a divalent metal cation (e.g., a calcium ion or a barium ion), or by cross-linking with a polycationic polymer (e.g., an amino acid polymer such as polylysine). See e.g., U.S. Pat. Nos. 4,806,355, 4,689,293 and 4,673,566 to Goosen et al.; U.S. Pat. Nos. 4,409,331, 4,407,957, 4,391,909 and 4,352,883 to Lim et al.; U.S. Pat. Nos. 4,749,620 and 4,744,933 to Rha et al.; and U.S. Pat. No. 5,427,935 to Wang et al Amino acid polymers that may be used to crosslink hydrogel forming polymers such as alginate include cationic poly(amino acids) such as polylysine, polyarginine, polyornithine, copolymers and blends thereof.

In certain embodiments, the core, the shell, or both the core and the shell comprise alginate or derivative thereof in combination with hyaluronic acid (HA), a cell (e.g., adult stem cell) and a protein (e.g., collagen or derivatives thereof or fibrin or derivatives thereof). In certain embodiments, the biocompatible, hydrogel-forming polymer used to form the shell is polyanionic compound, alginate or a derivative thereof. The hydrogel of the shell is referred to herein as a “shell hydrogel” and may include any of the hydrogel materials disclosed herein.

In some embodiments, the core, the shell, or both the core and the shell comprise hyaluronic acid, hyaluronan or derivatives thereof. Hyaluronic acid (HA) is a glycosaminoglycan present in many tissues throughout the body that plays an important role in embryonic development, wound healing, and angiogenesis. In addition, HA interacts with cells through cell-surface receptors to influence intracellular signaling pathways. Together, these qualities make HA attractive for tissue engineering scaffolds. HA can be modified with crosslinkable moieties, such as methacrylates and thiols, for cell encapsulation. Crosslinked HA gels remain susceptible to degradation by hyaluronidase, which breaks HA into oligosaccharide fragments of varying molecular weights. Auricular chondrocytes can be encapsulated in photopolymerized HA hydrogels where the gel structure is controlled by the macromer concentration and macromer molecular weight. In addition, photopolymerized HA and dextran hydrogels maintain long-term culture of undifferentiated human embryonic stem cells. HA hydrogels have also been fabricated through Michael-type addition reaction mechanisms where either acrylated HA is reacted with PEG-tetrathiol, or thiol-modified HA is reacted with PEG diacrylate.

In some embodiments, photocrosslinked chondroitin sulfate hydrogels can be integrated into the shell by modifying chondroitin sulfate with methacrylate groups. Hydrogel properties of the shell may be readily controlled by the degree of methacrylate substitution and macromer concentration in solution prior to polymerization. Further, the negatively charged polymer creates increased swelling pressures allowing the gel to imbibe more water without sacrificing its mechanical properties. Copolymer hydrogels of chondroitin sulfate and an inert polymer, such as PEG or PVA, may also be used.

In some embodiments, the core, the shell, or both the core and the shell comprise a synthetic polymer or polymers. In some embodiments, polyethylene glycol (PEG) is used to create macromers for cell encapsulation. A number of studies have used poly(ethylene glycol)di(meth)acrylate to encapsulate a variety of cells. Biodegradable PEG hydrogels can be prepared from triblock copolymers of poly(α-hydroxy esters)-b-poly(ethylene glycol)-b-poly(α-hydroxy esters) endcapped with (meth)acrylate functional groups to enable crosslinking PLA and poly(8-caprolactone) (PCL) have been the most commonly used poly(α-hydroxy esters) in creating biodegradable PEG macromers for cell encapsulation. The degradation profile and rate are controlled through the length of the degradable block and the chemistry. The ester bonds may also degrade by esterases present in serum, which accelerates degradation. Biodegradable PEG hydrogels can also be fabricated from precursors of PEG-bis-[2-acryloyloxy propanoate]. As an alternative to linear PEG macromers, PEG-based dendrimers of poly(glycerol-succinic acid)-PEG, which contain multiple reactive vinyl groups per PEG molecule, can be used. An attractive feature of these materials is the ability to control the degree of branching, which consequently affects the overall structural properties of the hydrogel and its degradation. Degradation will occur through the ester linkages present in the dendrimer backbone.

In embodiments, the biocompatible, a hydrogel-forming polymer can contain polyphosphoesters or polyphosphates where the phosphoester linkage is susceptible to hydrolytic degradation resulting in the release of phosphate. For example, a phosphoester can be incorporated into the backbone of a crosslinkable PEG macromer, poly(ethylene glycol)-di-[ethylphosphatidyl (ethylene glycol) methacrylate] (PhosPEG-dMA), to form a biodegradable hydrogel. The addition of alkaline phosphatase, an ECM component synthesized by bone cells, enhances degradation. The degradation product, phosphoric acid, reacts with calcium ions in the medium to produce insoluble calcium phosphate inducing autocalcification within the hydrogel. Poly(6-aminoethyl propylene phosphate), a polyphosphoester, can be modified with methacrylates to create multivinyl macromers where the degradation rate is controlled by the degree of derivatization of the polyphosphoester polymer.

In other embodiments, polyphosphazenes are used to cross-link compounds in the shell. Polyphosphazenes have a majority of side chain groups which are acidic and capable of forming salt bridges with di- or trivalent cations. Examples of preferred acidic side groups are carboxylic acid groups and sulfonic acid groups. Hydrolytically stable polyphosphazenes are formed of monomers having carboxylic acid side groups that are crosslinked by divalent or trivalent cations such as Ca²⁺ or Al³⁺. Polymers can be synthesized that degrade by hydrolysis by incorporating monomers having imidazole, amino acid ester, or glycerol side groups. Bioerodible polyphosphazenes have at least two differing types of side chains, acidic side groups capable of forming salt bridges with multivalent cations, and side groups that hydrolyze under in vivo conditions, e.g., imidazole groups, amino acid esters, glycerol, and glucosyl. Hydrolysis of the side chain results in erosion of the polymer. Examples of hydrolyzing side chains are unsubstituted and substituted imidazoles and amino acid esters in which the group is bonded to the phosphorous atom through an amino linkage (polyphosphazene polymers in which both R groups are attached in this manner are known as polyaminophosphazenes). For polyimidazolephosphazenes, some of the “R” groups on the polyphosphazene backbone are imidazole rings, attached to phosphorous in the backbone through a ring nitrogen atom.

In the preferred embodiment, the disclosed microcapsule compositions are adapted to simultaneously encapsulate cells within both the shell and the core. In addition, the cells in the shell can be distinct from the cells in the core. As described above, the composition of the core can be heterogenous, containing a mixture of scaffolds, solutions, cells, proteins, and other molecules. This is particularly useful for 3D culture and tissue engineering. For example, in some embodiments, the core could contain endothelial cells, and the shell could contain smooth muscle cells. In other examples, the shell and/or core may contain trophoblasts, adult stem cells, UV-sensitive compounds, a variety of crosslinking compounds, collagen, laminin, and the like. In one example, the core hydrogel/solution includes laminin, collagen, adult stem cells, and/or HA.

In embodiments, the core and shell can contain proteins, such as proteins suitable for promoting cell survival, attachment, growth, pluripotency, light activation, cell signaling, or differentiation. Further to the above, in some embodiments, the core, shell, or combination thereof further comprises fibrin, gelatin, elastin, or elastin-like polypeptides (ELPs), or a derivative thereof. In some embodiments, only the shell further comprises collagen, fibrin, gelatin, elastin, or elastin-like polypeptides (ELPs), or a derivative thereof. In some embodiments, the core further comprises collagen, fibrin, gelatin, elastin, or elastin-like polypeptides (ELPs), or a derivative thereof.

The cells or cell clusters encapsulated in the core and/or shell of the disclosed microcapsules can be any living cell type, including, but not limited to, keratinizing epithelial cells, wet stratified barrier epithelial cells, exocrine secretory epithelial cells, hormone-secreting cells, epithelial absorptive cells (gut, exocrine glands, and urogenital tract), metabolism and storage cells, barrier function cells (lung, gut, exocrine glands, and urogenital tract), epithelial cells lining closed internal body cavities, ciliated cells with propulsive function, extracellular core hydrogel/solution secretion cells, contractile cells, blood, and immune system cells, sensory transducer cells, autonomic neuron cells, sense organ and peripheral neuron supporting cells, central nervous system neurons and glial cells, lens cells, pigment cells, germ cells, and nurse cells. Also included are any stem cells and progenitor cells of the cells disclosed herein, as well as the cells they lead to. The cells can be pluripotent stem cells, multipotent stem cells, progenitor cells, primary cells, or gametes. The cells can be a mixture of single cells or cell clusters, such as antral or pre-antral follicles, or native tissue from other organs.

In certain embodiments, the cells are pluripotent cells, including both embryonic stem cells and induced pluripotent stem cells (iPSCs). In certain embodiments, the cells are mesenchymal stem cells, hematopoietic stem cells, fibroblasts, endothelial cells, pericytes, astrocytes, macrophages, or combinations thereof. In other embodiments, the cells are cardiac cells such as cardiac stem cells, cardiac spheroids, and/or beating cardiac spheroids. In certain embodiments, the cardiac cells (e.g., cardiomyocytes) express cardiomyocyte markers such as cardiac troponin T. In certain embodiments, the cells are cancer stem cells (CSCs), such as CSCs isolated from a tumor.

In embodiments, various types of cardiomyocytes (ventricular, atrial, sinus node, Purkinje, with pacemaker functions, etc.) may be encapsulated in the microcapsules disclosed herein. Notably, embryonic stem cells (“SC”) have the potential capability to generate these cardiomyocyte phenotypes in vitro but the yield is often insufficient. An alternative is to use adult SC (undifferentiated cells found in differentiated tissue that can proliferate, reproduce and differentiate into the specialized cell types of the tissues from which they were isolated). In other embodiments, SCs derived from bone marrow may be used as an attractive alternative. In embodiments, mesenchymal SC (MSC) of the bone marrow can be encapsulated via the methods disclosed herein, and thereafter differentiate into cardiomyocytes via standard 2D methods. In yet another embodiment, hematopoietic SC(HSC) of the bone marrow may be encapsulated, isolated, and expanded via the methods described herein. After release from the microcapsules via washing in an isotonic solution, the HSCs may be used to generate vascular epithelium, smooth muscle cells, and cardiomyocytes. In embodiments, adult SCs in the heart are ideal candidates for microencapsulation in that they need no reprogramming, give rise only to cells present in the heart, i.e. cardiomyocytes and vessels (endothelial cells and smooth muscles) and may, because this is their physiologic function, survive in transplant patients, integrate into the surrounding tissues and carry out their functions for longer periods without causing any damage. In other embodiments, a non-limiting list of additional stem cells that may be encapsulated, isolated, and expanded via the herein disclosed microcapsules include adult stem cells (e.g., hematopoietic SCs, blood SCs, mesenchymal SCs, neural SCs, epithelial SCs, skin SCs, adipose SCs) totipotent SCs, omnipotent) SCs, pluripotent SCs, multipotent SCs, oligopotent SCs, unipotent SCs, tissue-specific stem cells, and mesenchymal stem cells. In embodiments, the disclosure provides for microencapsulation of any cell with proliferative capacity, including mammalian SCs, aquatic SCs, human SCs, and the like.

In certain aspects, the disclosed microcapsule compositions are unique in their ability to encapsulate cells within an aqueous, heterogenous liquid. For example, pluripotent stem cells can be encapsulated in an aqueous, heterogenous liquid, such as a cellulose solution including other proteins, or such as a carboxymethyl cellulose solution including other proteins and compounds and cultured to maintain pluripotency.

The disclosed microcapsules (e.g., core, shell, or combinations thereof) can further contain one or more bioactive agents within the core, shell, or combination thereof. In some embodiments, the bioactive agent is a therapeutic agent. In some embodiments, the bioactive agent can be a biomolecule. In certain embodiments, the bioactive agent can be a differentiation agent, such as a growth factor or chemokine suitable to stimulate the growth, survival, pluripotency, or differentiation of the cells encapsulated within the microcapsules. In certain embodiments, the bioactive agent is a growth factor such as VEGF (vascular endothelial growth factor), FGF (fibroblast growth factor), TGF (transforming growth factor), or combinations thereof. In some embodiments, the bioactive agent is a therapeutic agent such as an immunosuppressant and/or an anti-inflammatory agent.

In embodiments, the disclosed microcapsules can have an average diameter ranging from about 50 microns to about 2000 microns, including about 100 microns to about 100 microns, and about 30 microns to about 300 microns. In some embodiments, the shell has an average thickness greater than 10, 20, 30, 40, 50, 100, 200, 300, 400, 500, or 600 microns. For example, the shell can have an average thickness ranging from about 10 microns to about 100 microns, including about 50 microns.

In some embodiments, the volume of the core can have an average volume ranging from about 0.005 nanoliters to about 500 nanoliters, including about 0.1 nanoliters to about 50 nanoliters. In other embodiments, the volume of the core can have an average volume ranging from about 8 nanoliter to about 12 nanoliter, including about 9 nanoliter to about 11 nanoliter. In the preferred embodiment, the volume of the core has an average volume ranging from 10.1±0.9 nanoliter. For example, at the flow rate given in Table 1, the volume of the core with a diameter of 268.7±27.5 μm is ˜10 (10.1±0.9) nanoliter on average. Since the diffusion limit for oxygen and nutrients in a highly cellularized tissue is less than ˜200 μm^{[19]}, the small size (<˜200 μm in radius) of the core-shell microcapsules allows adequate mass transport for all the encapsulated cells to survive and proliferate. In other embodiments, a volume of 8-12 nanoliters (preferably ˜10 nanoliters) for the Zona-like shell is sized such that the highly pluripotent human iPSC clusters maintain a suitable microenvironment by releasing autocrine and/or paracrines inside the microcapsule for their survival/growth into highly pluripotent 3D spheroids^{[15d-g,21]}. In one example, the stem cell encapsulated by the microcapsule is an iPSC and/or iPSC cluster, and the size of the Zona-like shell permits the iPSC and/or iPSC cluster to release autocrine and other biomolecules inside the microcapsule, thereby enhancing the health of the iPSC and/or iPSC cluster.

Methods of Forming Microcapsules

Disclosed is a novel method for formation of a microcapsule including encapsulating a cell (e.g., a stem cell or stem cell cluster, human iPSC or iPSC cluster, CSC, and the like) thereby inducing isolation and expansion of the cells with improved homogeneity, efficiency, quality, and pluripotency.

Disclosed are methods for formation of microcapsules that encapsulate cells (e.g., stem cells, stem cell clusters, “blastomere cluster-like” human iPSC clusters, cancer stem cells, and the like) in a ˜10-nanoliter HA-rich hydrogel core of microcapsules with a “Zona-like” semipermeable hydrogel shell for isolation/purification and culture of highly pluripotent human iPSCs. Also provided are methods for improving differentiation, improving isolation, increasing efficiencies, and increasing homogeneity. Also provided are methods for isolating and expanding human iPSCs or human iPSC clusters including steps of encapsulating human iPSC clusters in a HA-rich core enclosed in a shell comprising an alginate hydrogel and/or other bioactive agents.

The disclosed methods benefit from several significant and surprising advantages including, but not limited to: a) substantially lower cost relative to standard 2D and 3D methods, b) highly homogenous cell populations, c) high pluripotency, d) unique scalability, e) lower requirement for skilled labor (e.g., no manual separation of dissociated cells necessary), f) improved cell isolation (e.g., enabling, for example, the isolation and expansion of a single cancer stem from a heterogenous tumor mass), g) significantly improved differentiation in 2D following growth in the 3D microculture, and h) significant overall improvements in culturing efficiency.

In embodiments, microencapsulation and “proliferation-only” growth of cells in the miniaturized 3D microenvironment of the HA-rich microcapsule core has the effect of improving isolation and improving the efficiency of expansion of high-quality human iPSC from heterogeneous 2D-cultured human iPSCs clusters. In the preferred embodiment, the microencapsulated human iPSC clusters can proliferate into “ICM-like” spheroids inside the microcapsules in ˜4 days of miniaturized culture, which is similar to the time for a 2-16 cell-stage blastomere cluster to grow into an ICM (3-5 days) naturally in the female reproductive system of humans. Moreover, the human iPSCs cultured inside the early embryo-like microcapsules have both significantly higher expression of pluripotency markers and capacity of 3D cardiac differentiation into beating cardiomyocytes than the human iPSCs from the conventional 3D suspension culture. Collectively, the bioinspired 3D microencapsulation culture of human iPSCs is able to isolate the highly pluripotent human iPSCs from a typically heterogeneous human iPSC population of 2D culture, which is invaluable for obtaining high-quality human iPSCs to facilitate the success of the human iPSC-based personalized disease modeling and regenerative medicine.

As described above, the methods disclosed herein use bioinspired microencapsulation and nanoscale culture conditions for the establishment of highly homogenous, high-quality, and highly pluripotent iPSCs. The highly pluripotent iPSCs may be released from microcapsules for further culturing in 2D while maintaining high pluripotency. Said highly pluripotent iPSCs are easy to propagate, and maintain long-term stable morphology, karyotype, and pluripotency marker expression typical of pluripotent cells. Subsequently culturing the cells in vitro indefinitely allows for introducing desired genetic variants by gene editing or traditional genetic engineering. From a biomedical perspective, the derivation of stable iPSCs is also important for genomic testing and selection, genetic engineering, and providing an experimental tool for studying human diseases.

In embodiments, the hydrogel microcapsules include a Zona-like hydrogel shell. As described above, said shell is generated to encapsulate a “blastomere cluster-like” iPSC cluster. In embodiments, said iPSC cluster may include a human iPSC or human iPSC cluster of 3-13 cells in each of the HA-containing microcapsule cores for culture, in a high-throughput manner using a non-planar microfluidic device. Notably, the core may include a heterogenous mixture of aqueous solution, proteins, compounds, and/or cells.

In some embodiments, a nanoliter-scale and HA-rich microenvironment enclosed in the semipermeable “Zona-like” hydrogel shell allows the blastomere cluster-like human iPSC clusters to grow into “inner cell mass (ICM)-like” human iPSC spheroids that are highly pluripotent. The herein disclosed data show that the hydrogel core-shell microcapsule with a HA-rich nanoliter core is crucial to selectively isolate a subset of human iPSC clusters from conventional 2D culture to proliferate into the ICM-like human iPSC spheroids within 3-5 days (preferably 4 days) of the bioinspired 3D culture. In embodiments, a subset of human iPSCs possess higher levels of pluripotency markers than that from traditional 3D suspension and 2D cultures. Therefore, this bioinspired culture can effectively isolate high-quality human iPSCs in a high-throughput manner.

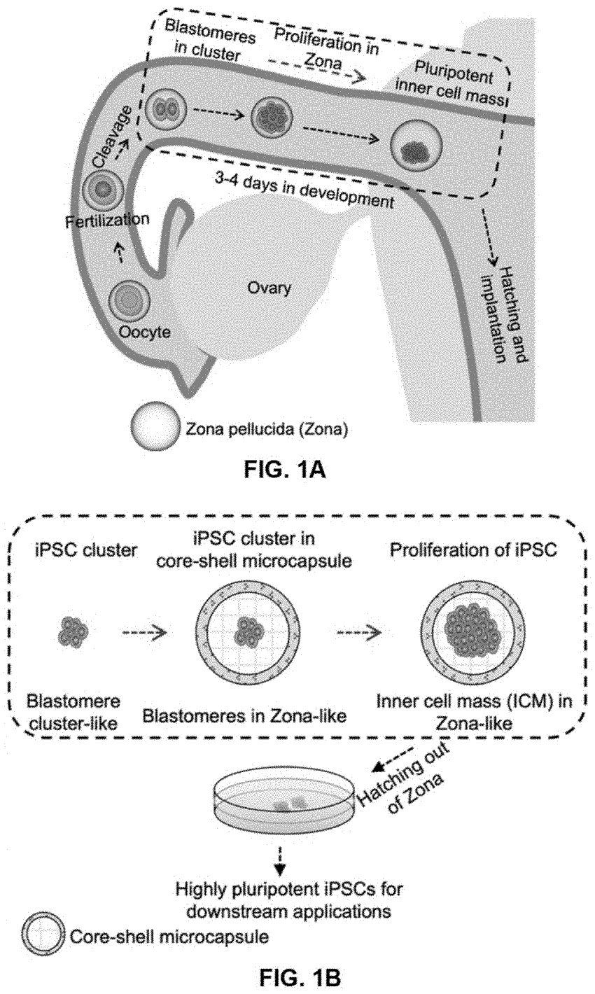

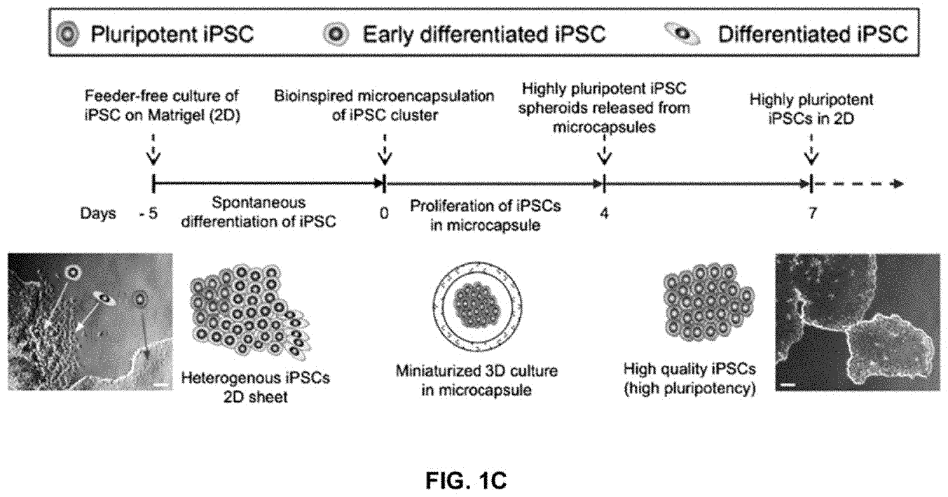

A model female reproductive system is depicted in FIG. 1A wherein ESCs contained in the ICM are developed from blastomeres (e.g., at the 2 to 16-cell stages) via growth/proliferation without merging/fusion with blastomeres in any other early embryos^{[15e,17]}. In embodiments, this procedure includes the following key features: 1) encapsulation of a cluster of multiple blastomeres in a nanoliter core that is enclosed in a semipermeable Zona shell, and 2) proliferation of the blastomeres in the miniaturized core for 3-4 days to produce the ESC-containing ICM before hatching out of the Zona shell for implantation. In embodiments, the present disclosure provides a strategy which has similarities to the basic steps of the aforementioned procedure in ICM/ESC development.

In embodiments, as shown in FIG. 1B, the herein disclosed methods can be used to isolate and culture highly pluripotent human iPSCs among a conventionally cultured heterogeneous human iPSC population. In embodiments, “blastomere cluster-like” human iPSC clusters and fabricated “Zona-like” core-shell microcapsules are produced with a nanoliter-scale hydrogel core enclosed in a semipermeable alginate hydrogel shell. In the preferred embodiment, one “blastomere cluster-like” human iPSC cluster is encapsulated in each of the microcapsules for proliferation-only culture. In embodiments, after ˜4 days of proliferation, only the highly pluripotent individual human iPSC clusters encapsulated in the Zona-like microcapsules can grow into an “ICM-like” 3D human iPSC spheroid in each microcapsule (FIG. 1B-1C). This growth and the human iPSCs mimic the aforementioned formation of the ESCs-containing pluripotent ICM in the pre-hatching embryos in vivo. FIG. 1C shows that, after ˜3-5 days of proliferation (preferably ˜4 days), the highly pluripotent individual human iPSC clusters encapsulated in the Zona-like microcapsules grow into an “ICM-like” 3D human iPSC spheroid in each microcapsule.

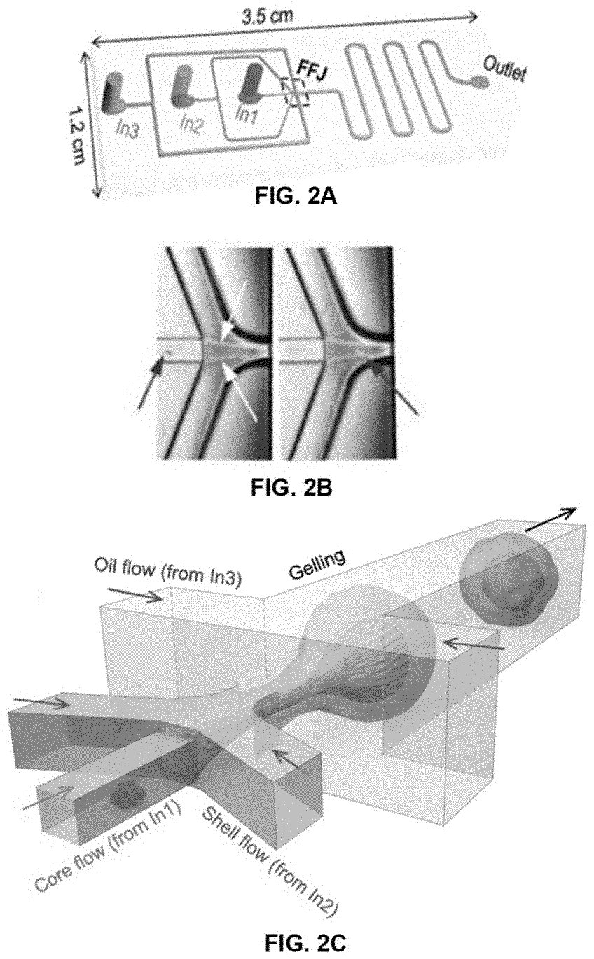

As depicted in FIG. 2A-2C, in the preferred embodiment the core-shell microcapsule (also referred to herein as “microcapsule”) is produced in a single step. In other embodiments, multiple coating steps can also be used to prep microcapsules via conventional methods, which in some embodiments may be facilitated by the use of polyionic materials. In one embodiment, a microcapsule includes a core comprising a cell (e.g., a stem cell, iPSC, mammalian cell) or cell aggregate (e.g., iPSC cluster) suspended or encapsulated in a core hydrogel/solution (e.g., a solution including sodium alginate, HA, and the like). The microcapsule may further include a shell surrounding the core comprising a biocompatible hydrogel, wherein the core hydrogel/solution and biocompatible hydrogel are formed from materials with different chemical compositions in some embodiments. In some embodiments, the core contains a heterogenous mixture of proteins, scaffolds, small molecules, UV-active molecules, cells and/or various solutions.

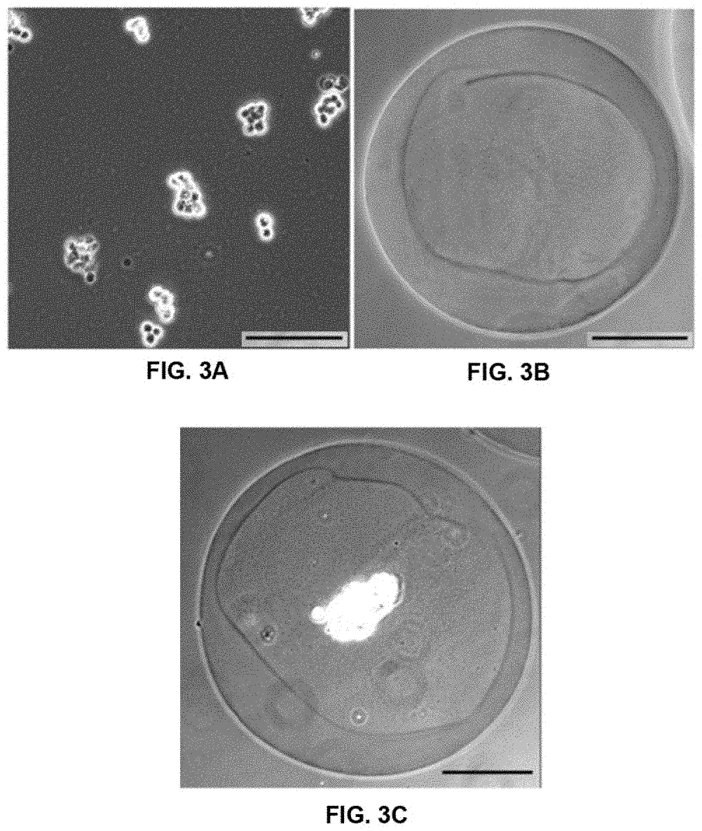

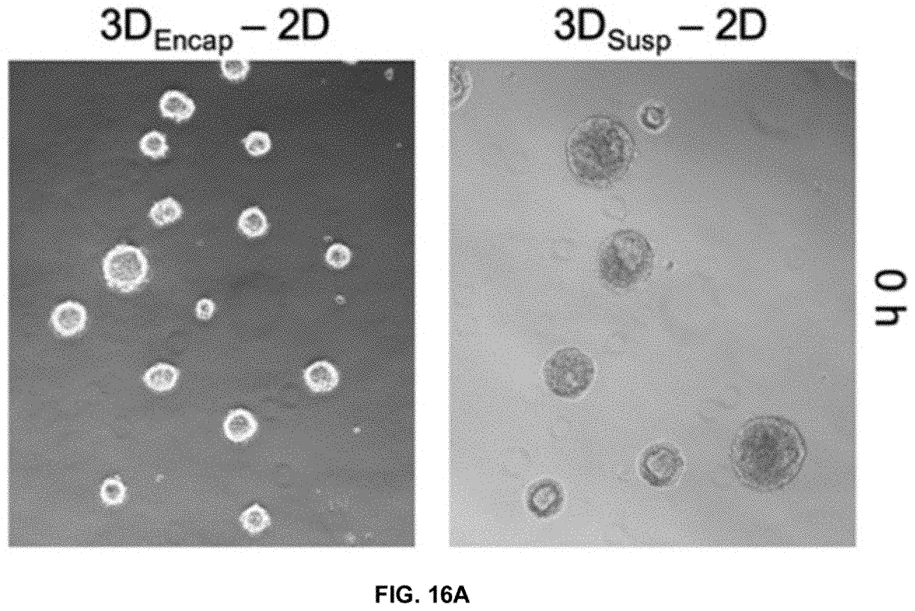

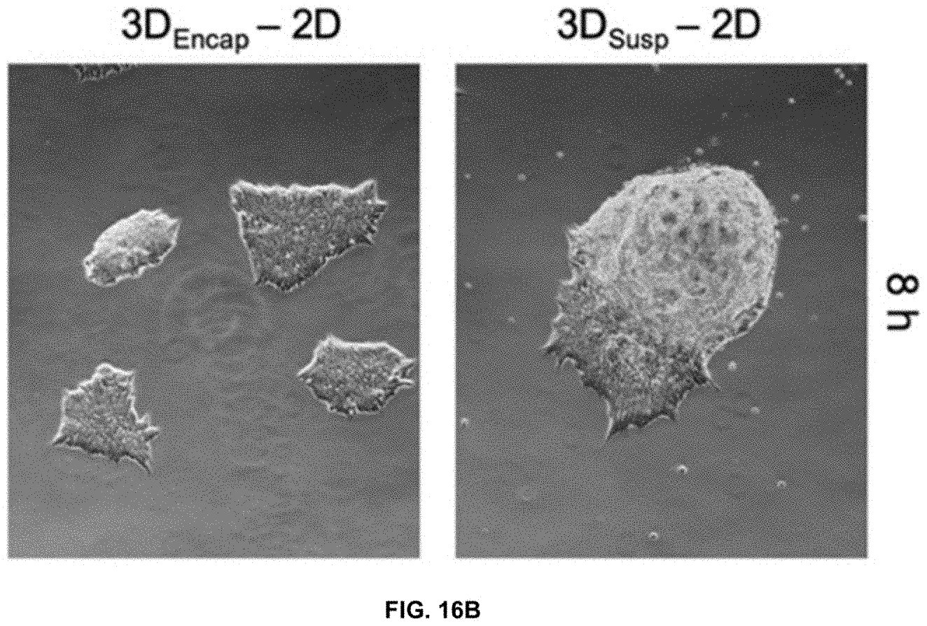

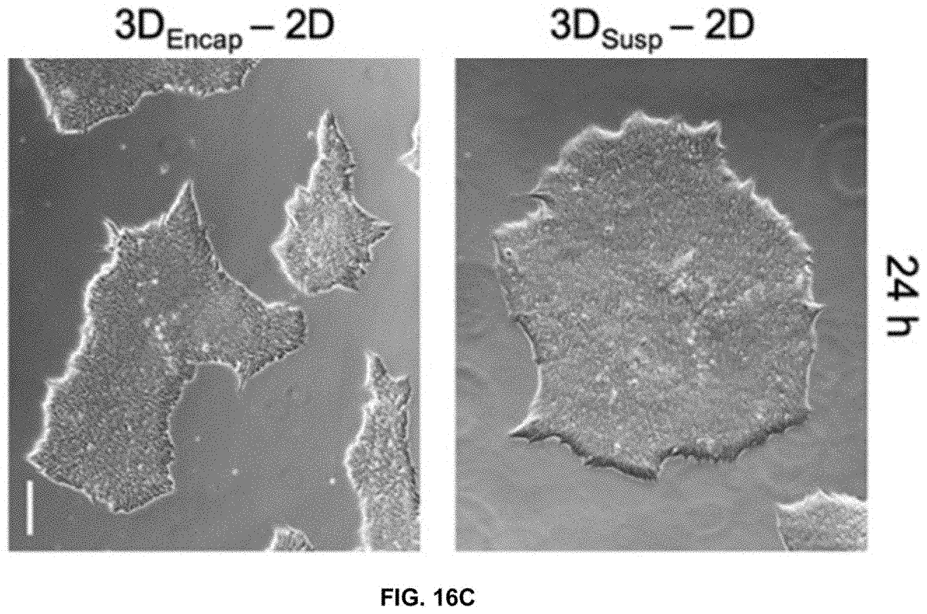

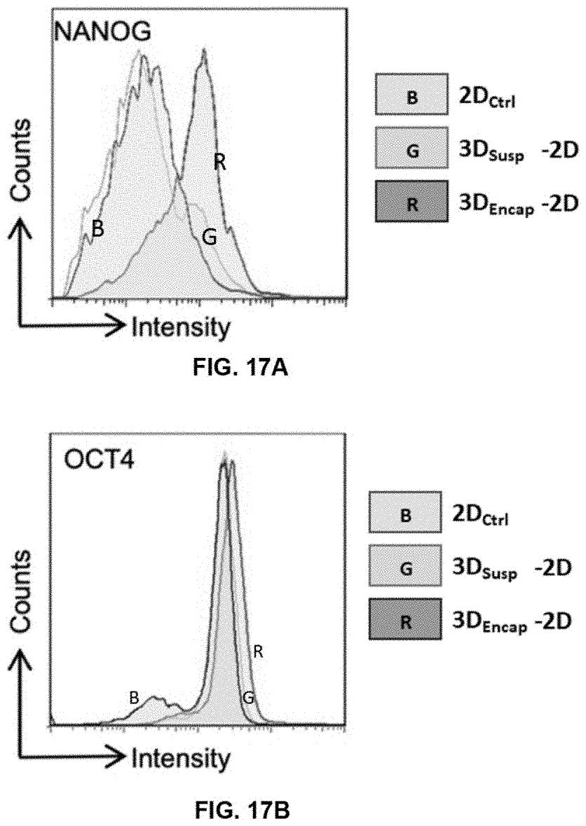

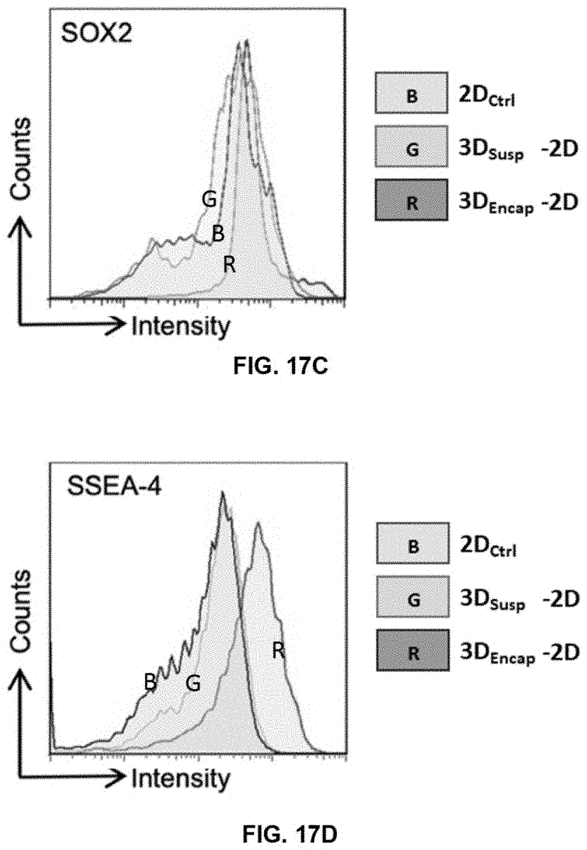

In some embodiments, the highly pluripotent “ICM-like” human iPSC spheroids are adapted to be conveniently released from the microcapsules for further use with standard 2D culture methods as shown in FIG. 16A-16C. In embodiments, said spheroids are released by liquifying the alginate hydrogel using an isotonic solution (e.g., phosphate-buffered saline or “PBS”, which is isotonic by default). As shown in FIG. 17A-17D, pluripotent human iPSC spheroids may be cultured in 2D while maintaining high pluripotency. FIG. 3A depicts stem cells or stem cell clusters (e.g., iPSC clusters), FIG. 3B shows a Zona-like microcapsule, and FIG. 3C shows the final microcapsule product.

In embodiments, the “blastomere cluster-like” human iPSC clusters (“iPSC clusters”) include an average of 3-13 cells per cluster (See FIG. 4A). In other embodiments, the iPSC clusters include an average of 4-5 cells per cluster, 3-7 cells per cluster, 3-11 cells per cluster, 2-15 cells per cluster, or 1-20 cells per cluster. In embodiments, said iPSC clusters are generated via a method including the steps of a) detaching 2D cultured human iPSC monolayers, b) gently pipetting the detached monolayers into small clusters, then c) encapsulating the clusters in the “Zona-like” core-shell microcapsules (See FIG. 3A-3C) using the above-mentioned microfluidic device.

Also disclosed are methods for producing alginate microcapsules having controlled dimensions. For example, microcapsules may be monodispersed in size (see FIG. 4B). In some embodiments, microcapsules are produced with average diameters of less than 10%, 15%, 20%, 25%, or 30% variation.

Microencapsulated cells produced by the disclosed methods may be used for miniaturized 3D culture and differentiation of the cells, cryoprotection (e.g., cryopreservation), cell-based drug delivery, cell transplantation, tissue regeneration, personalized medicine, and assisted reproductive medicine. For example, in addition to a potential role in early diagnosis and prognosis, improvements in the detection of CSCs enabled by biomimetic microencapsulation can guide therapeutic strategies for personalized treatment of patients with metastatic cancer.

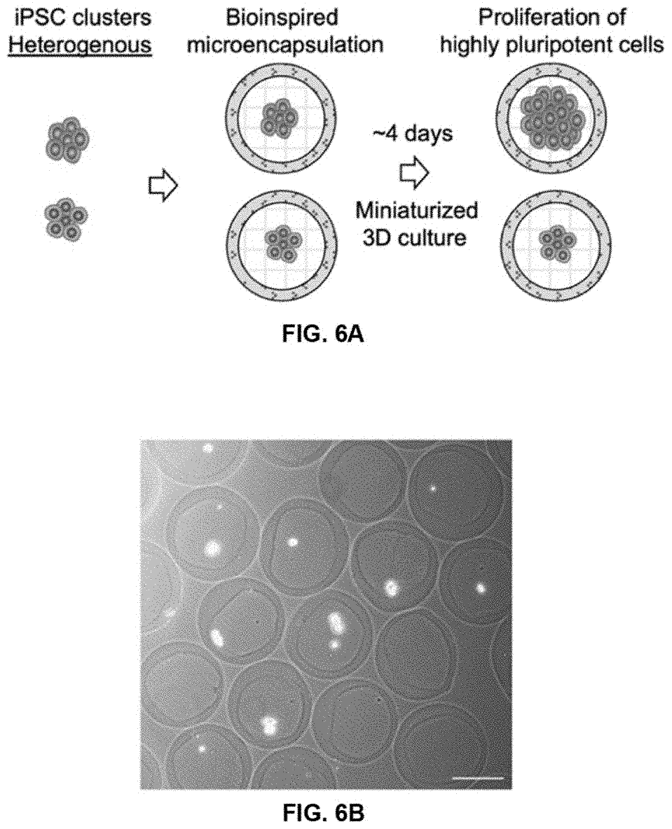

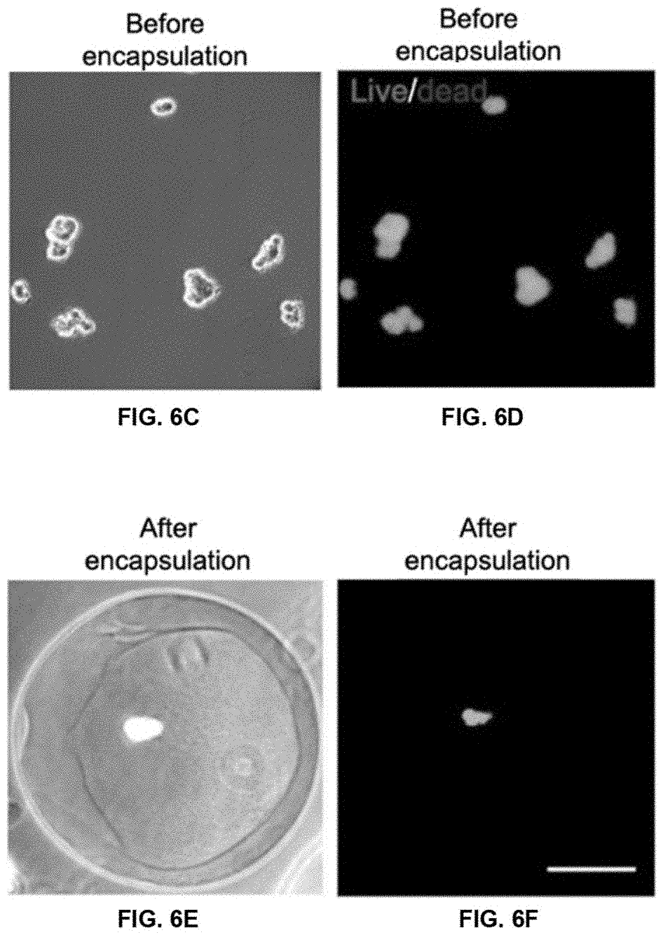

In embodiments, use of the herein disclosed microencapsulation method creates a miniaturized HA-rich 3D microenvironment for cells or cell clusters (e.g., mammalian stem cells, human iPSC clusters, cancer stem cells, cardiac stem cells, and the like) to proliferate into “ICM-like” human iPSC spheroids. In some embodiments, after 3 to 5 days of miniatured culture (preferably, 4 days), like the proliferation of human blastomeres into ICM during early embryonic development, the cells (e.g., stem cells, highly pluripotent human iPSCs, and the like) can proliferate in the Zona-like and HA-rich core-shell microcapsules (See FIG. 6A). Typical morphologies of human iPSC clusters before and after microencapsulation are shown in FIG. 6B-6F.

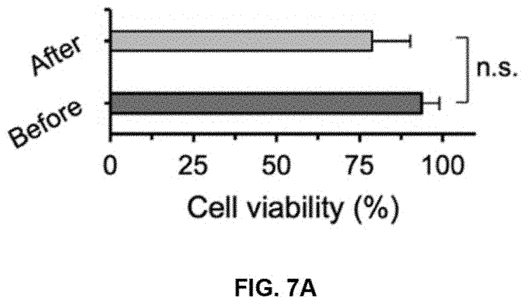

In embodiments, although there are some empty microcapsules (i.e., microcapsules without any cells in their core) forming during the encapsulation process, the presence of empty microcapsules does not affect cell culture. Further, the empty microcapsules can be easily liquified using PBS when releasing and collecting the human iPSC spheroids for further uses. On the other hand, the group has recently developed platforms to selectively collect microcapsules with cells or clusters of interest by using one or more optical sensors or deep learning-based image processing for label-free detection of cell/cluster-laden hydrogel microcapsules in combination with dielectrophoretic forces for on-chip manipulation of the microcapsules. These technologies would certainly be beneficial to the future use of the presented work when samples of highly pure cell-laden microcapsules are required. The human iPSC clusters are highly viable both before and after encapsulation in the core of each microcapsule with no statistically significant difference, indicating the biocompatible/mild nature of the microfluidic encapsulation, gelling, and washing processes involved in generating the human iPSC-laden microcapsules (See FIG. 6C-6F and FIG. 7A). FIG. 7A shows the quantitative analysis associated with FIG. 6C-6F tracking cell viability before and after encapsulation.

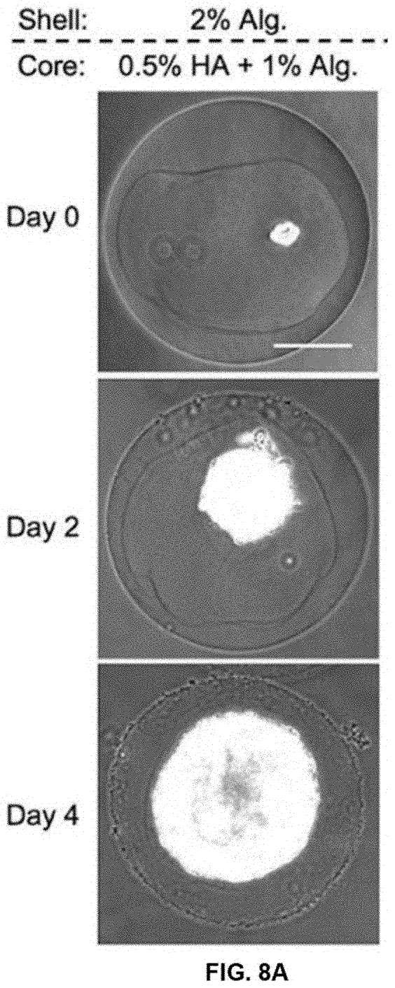

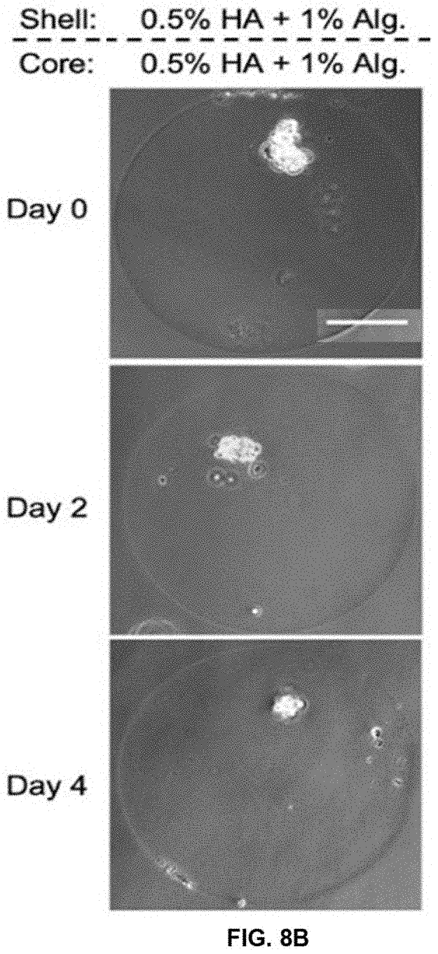

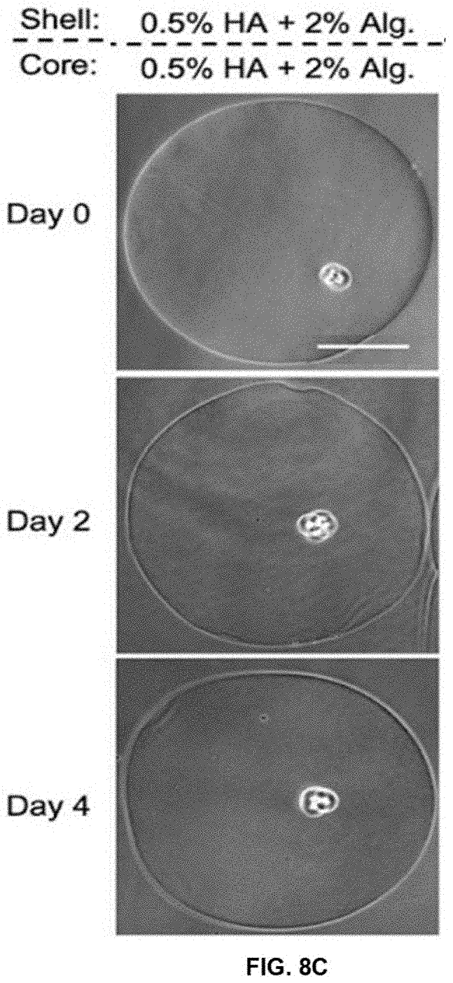

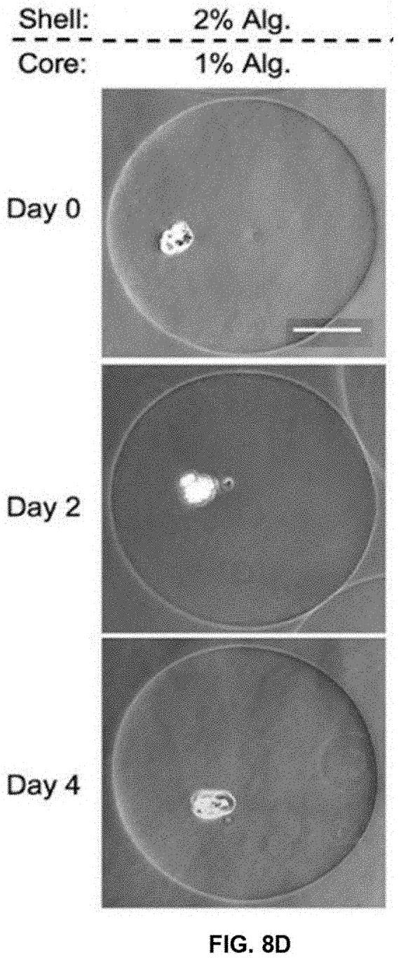

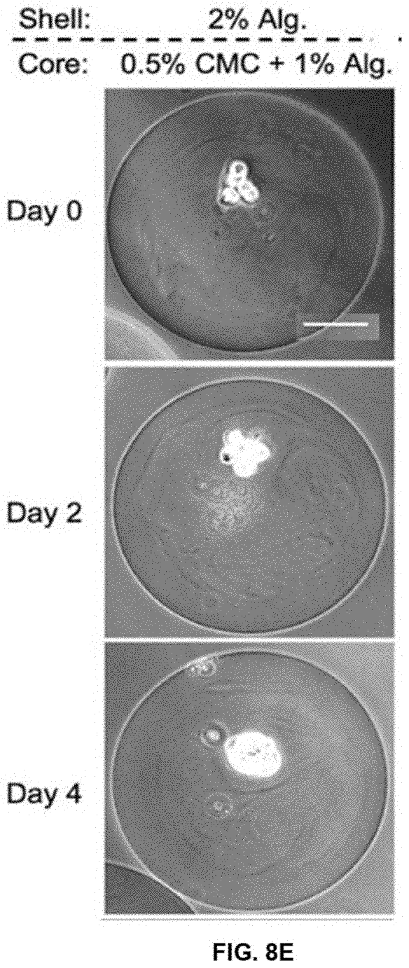

In some embodiments, a short (˜30 min in total) exposure of the human iPSCs to calcium ions during the encapsulation process is not harmful (e.g., because the exposure is at ice temperature with negligible cellular activities) and the mineral oil is not damaging to the cells either (e.g., because it has no direct contact with the human iPSCs in the core of the microcapsules). In another embodiment, after 4 days of culture, a subset (˜20%) of the human iPSC clusters can proliferate into spheroids in the HA-rich nanoliter core of the microcapsules. Representative images showing the proliferation are given in FIG. 8A-8E, including phase contrast images showing the proliferation of human iPSC clusters over 4 days in the core of the HA-rich core-shell microcapsules (“microcapsules”). The images include shell conditions of 2% alginate plus core conditions of 0.5% HA+1% alginate (See FIG. 8A), shell conditions of 0.5% HA+1% alginate with equal core conditions (See FIG. 8B), shell conditions of 0.5% HA+2% alginate with equal core conditions (See FIG. 8C), shell conditions at 2% alginate plus core conditions of 1% alginate (See FIG. 8D), and shell conditions at 2% alginate plus core at 0.5% CMC+1% alginate (FIG. 8E).

In some embodiments, the stem cells or stem cell clusters (e.g., human iPSCs, cardiac stem cells, cancer stem cells, and the like) are encapsulated in alginate hydrogel microbeads without a core-shell structure using the following culture conditions: (1) microbeads of 1% alginate (used in the core of the core-shell microcapsules) and 0.5% HA (FIG. 8B), and (2) microbeads of 2% alginate (used in the shell of the core-shell microcapsules) and 0.5% HA (FIG. 8C). Notably, the 2% alginate hydrogel shell is semipermeable, which permits adequate transport of oxygen and nutrients via diffusion for stem cells (e.g., human iPSCs) to survive and proliferate (FIG. 8A). It also provides a physical barrier for achieving proliferation-only culture of the human iPSCs. In embodiments, the 2% alginate hydrogel shell performs a similar function to the zona pellucida hydrogel shell in early embryos, viz., to maintain the nanoliter core of HA and possibly autocrines and/or paracrines in the microcapsules/early embryos, although their compositions are not the same. In embodiments, stem cells or stem cell clusters (e.g., human iPSC clusters) may be encapsulated without HA in the core (See FIG. 8D). Under these conditions, no evident core-shell structure can be seen for the microcapsules made in the absence of HA, possibly due to a similar optical property of the 1-2% alginate hydrogel and the mixing of the 1% alginate (less viscous than the solution of 1% alginate+0.5% HA) flow with the 2% alginate flow during the microfluidic encapsulation. In the preferred embodiment, sufficiently high viscosity of all aqueous fluids has been shown to be critical for generating microcapsules with a hydrogel (aqueous) core and hydrogel (aqueous) shell configuration^{[25]}. In some embodiments, HA is replaced with 0.5% carboxymethyl cellulose (CMC) in the core fluid to fabricate core-shell microcapsules for human iPSC microencapsulation (without HA) (See FIG. 8E). Together, these data (See FIG. 7A, FIG. 8A, FIG. 8B, FIG. 8C, FIG. 8D, and FIG. 8E) indicate the critical importance of the HA-rich and nanoliter-sized core in bioinspired 3D microencapsulation culture system for efficiently isolating the human iPSCs in 3-5 days (preferably, 4 days).

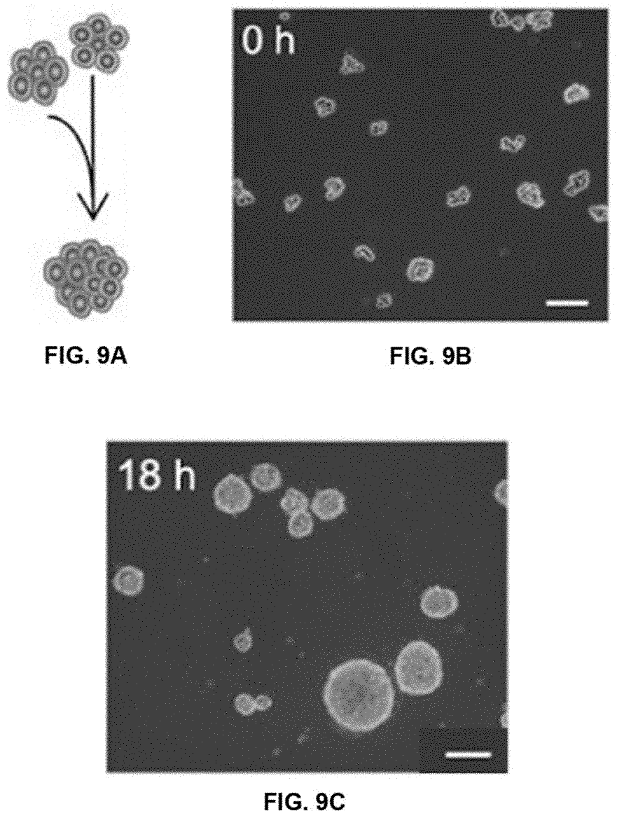

To generate human iPSC spheroids, the present methods are also compatible with concurrent use of conventional 3D suspension culture^{[26]}. For generating human iPSC spheroids of comparable size (on average), conventional 3D suspension culture is a fast option (in ˜18 h, FIG. 9A-9C). FIG. 9B shows phase contrast image at time zero while FIG. 9C is a phase contrast image at time 18 hours. In embodiments, the herein disclosed bioinspired microencapsulation approach requires 4 days on average, by contrast. In embodiments, the human iPSC spheroids from the 3D suspension culture method form through merging/fusion of multiple human iPSC clusters and/or spheroids^{[6e]}, whereas the human iPSC spheroids from the microencapsulation approach form through proliferation. FIG. 9A-9C are schematic illustrations and representative phase contrast images showing the formation of human iPSC spheroids under conventional suspension culture, in which aggregation/fusion of human iPSC clusters/spheroids may occur. FIG. 9A is an illustration depicting aggregation/fusion of human iPSC clusters/spheroids.

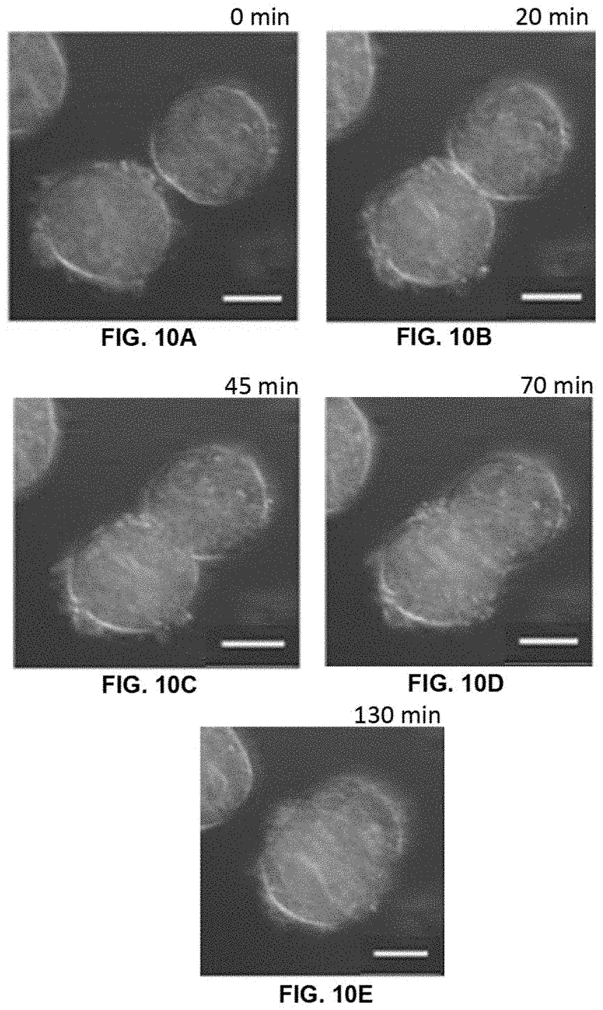

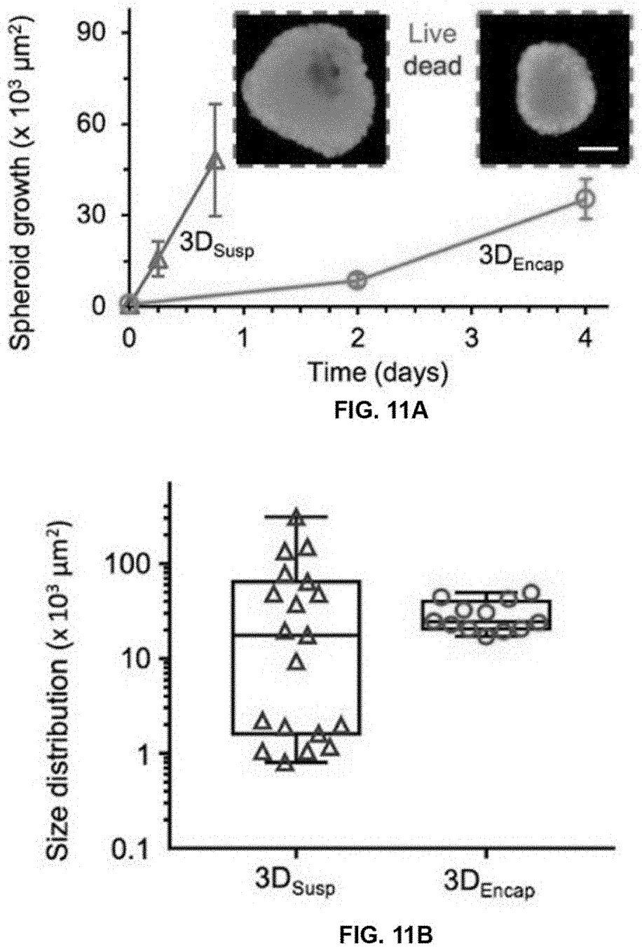

As shown in FIG. 10A-10E, in some embodiments smaller human iPSCs spheroids are adapted to fuse into a larger spheroid quickly in only ˜2 h, showing that the fast “growth” of human iPSC spheroids under the conventional 3D suspension culture is due to merging/fusing. This uncontrolled merging/fusion may result in heterogenous human iPSC spheroids in diameter. FIG. 10A-10E show time-sequence images show two smaller human iPSC spheroids fusing into a larger spheroid under the conventional suspension culture at time zero (See FIG. 10A), 20 minutes (See FIG. 10B), 45 minutes (See FIG. 10C), 70 minutes (See FIG. 10D), and 130 minutes (See FIG. 10E). Although both methods can effectively form highly viable human iPSC spheroids as indicated by live/dead staining analyses (See FIG. 11A), the human iPSC spheroids from the conventional 3D suspension culture have a wide size distribution of 30-500 μm in diameter. In contrast, the human iPSC spheroids from the proliferation-only culture in the core-shell microcapsules have a much narrower size distribution of 80-180 μm in diameter (See FIG. 11B). FIG. 11A shows the size change of human iPSC spheroids over time under both the conventional 3D suspension (3DSusp) culture and microencapsulation (3DEncap) culture. The inset images of live/dead (green/red) staining show both culture methods can effectively form highly viable human iPSC spheroids. FIG. 11B shows the size distributions of human iPSC spheroids obtained from the conventional 3D suspension culture and bioinspired 3D microencapsulation culture.

In embodiments, the high viability of all the spheroids demonstrates that there is no lack of oxygen and nutrient diffusion into the spheroid core, again probably because their radius is less than the diffusion length (˜200 μm) of oxygen and nutrients in highly cellularized tissues^{[19]}. In one embodiment, a human iPSC cluster proliferates into a highly pluripotent iPSC spheroid within the microcapsule, the highly pluripotent iPSC spheroid having a radius of less than 500 microns. Moreover, the uncontrolled nature of the merging/fusion of human iPSC clusters/spheroids indicates the spheroids from the conventional 3D suspension culture may retain the heterogeneity of the human iPSCs from 2D culture. In embodiments, unlike the conventional 3D suspension method, the “Zona-like” hydrogel shell prevents merging/fusing between adjacent clusters/spheroids. This allows for the human iPSC clusters in the microcapsules to grow only through proliferation (See FIG. 6A).

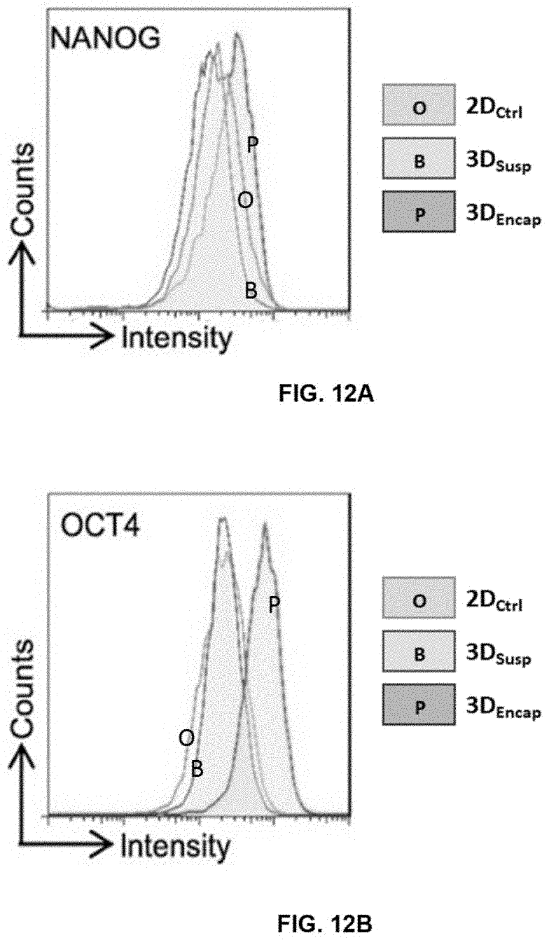

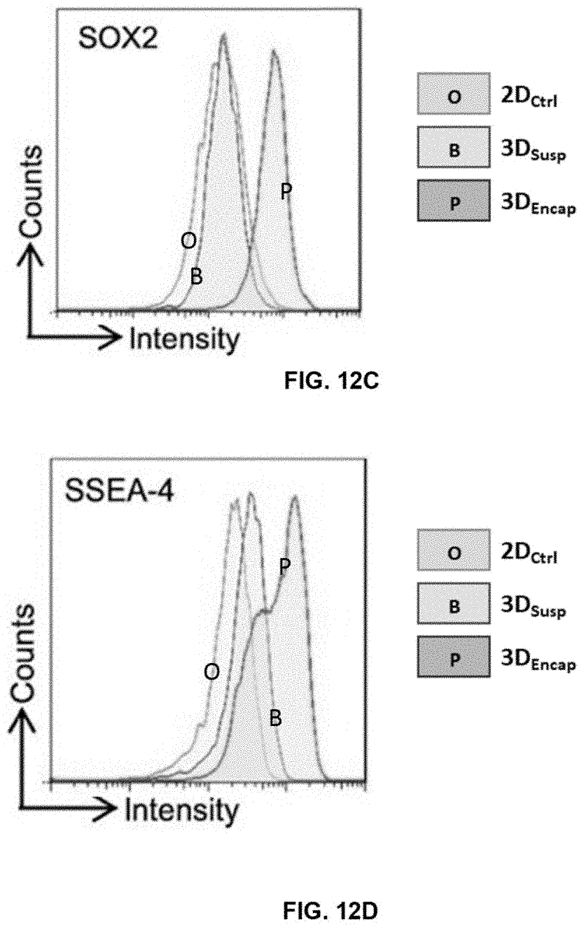

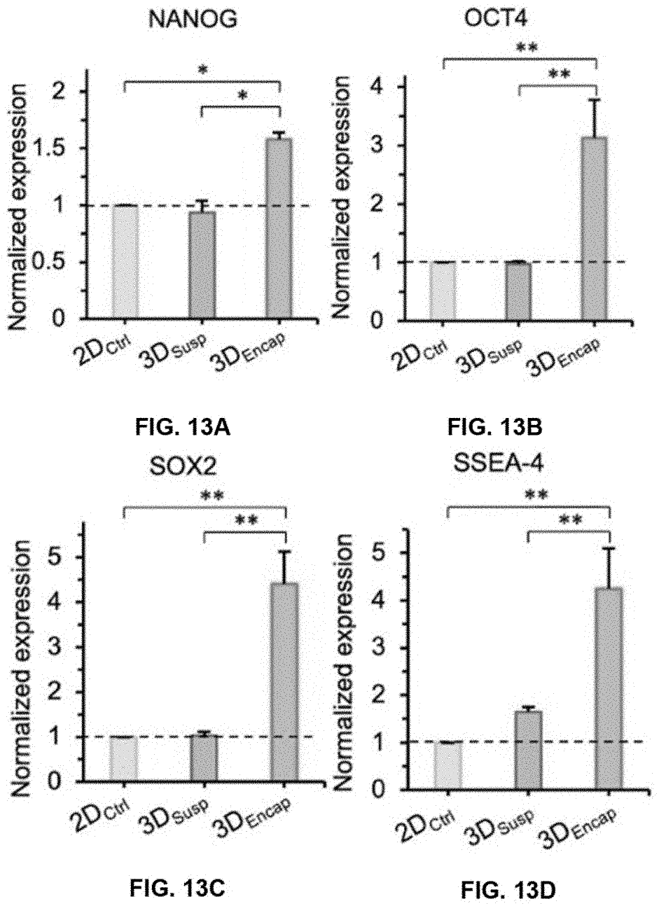

In some embodiments, the proliferation time (˜4 days) for the subset (˜20%) of human iPSCs to grow into spheroids is similar to the proliferation of blastomeres into ICM (3-5 days) during early embryonic development of humans. Similar to the human ESCs from the ICM of an embryo, the microencapsulated human iPSC spheroids are highly pluripotent. In embodiments, the key pluripotency markers (NANOG, OCT4, SOX2, and SSEA-4) are used to evaluate the quality of human iPSCs. Human iPSC spheroids from conventional 3D suspension culture are used for comparison. FIG. 12A shows flow cytometry peaks for NANOG (See FIG. 12A), OCT4 (See FIG. 12B), SOX2 (See FIG. 12C), and SSEA-4 (See FIG. 12D) in human iPSCs under bioinspired 3D microencapsulation culture (“P”) versus conventional 2D and 3D suspension cultures (“O” and “B”, respectively). According to flow cytometry analyses (See FIG. 13A-13D), the expression of these pluripotency markers is not significantly different when comparing the human iPSCs from the conventional 3D_{Susp} with 2D control (2D_{Ctrl}) cultures.

In embodiments, the aforementioned data shows that the conventional 3D_{Susp} culture retains the heterogeneity of the human iPSCs from 2D culture, probably due to the nature of the suspension culture method which lacks a selection or isolation mechanism. In this case, the human iPSCs spheroids form through uncontrolled merging/fusion of adjacent heterogeneous clusters/spheroids (See FIG. 9A-9C and FIG. 10A-10E). In contrast, human iPSCs from the disclosed bioinspired 3D_{Encap} culture have significantly higher expression of pluripotency markers than the cells from both conventional 2D and 3D_{Susp} culture methods. Quantitative analyses show that the expression of NANOG, OCT4, SOX2, and SSEA-4 in the human iPSCs from the bioinspired 3D_{Encap} culture is 1.6 (1.7)-fold, 3.1 (3.1)-fold, 4.4 (4.3)-fold, and 4.2 (2.6)-fold higher, respectively, than that in the human iPSCs from the conventional 2D (3D_{Susp}) cultures (FIG. 13A-13D). FIG. 13A-13D shows the quantitative analysis corresponding to the data provided in FIG. 12A-12D, including analysis for NANOG (FIG. 13A), OCT4 (FIG. 13B), SOX2 (FIG. 13C), and SSEA-4 (FIG. 13D). For the quantitative analysis, the peak intensity for the two 3D culture conditions is normalized to that for the 2D culture. Data are presented as mean±standard deviation (s.d.). One-way analysis of variance (ANOVA) with Tukey's post hoc analysis was used to assess statistical significance, *p<0.05, **p<0.01.

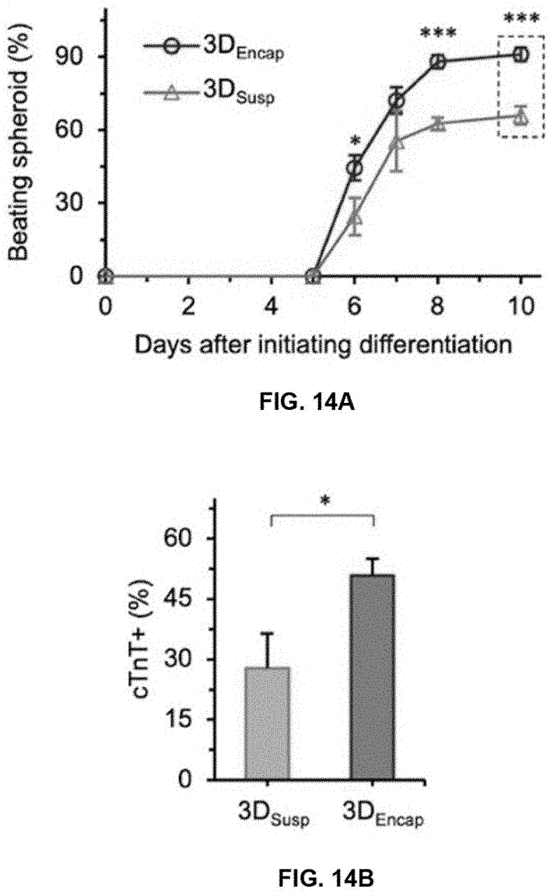

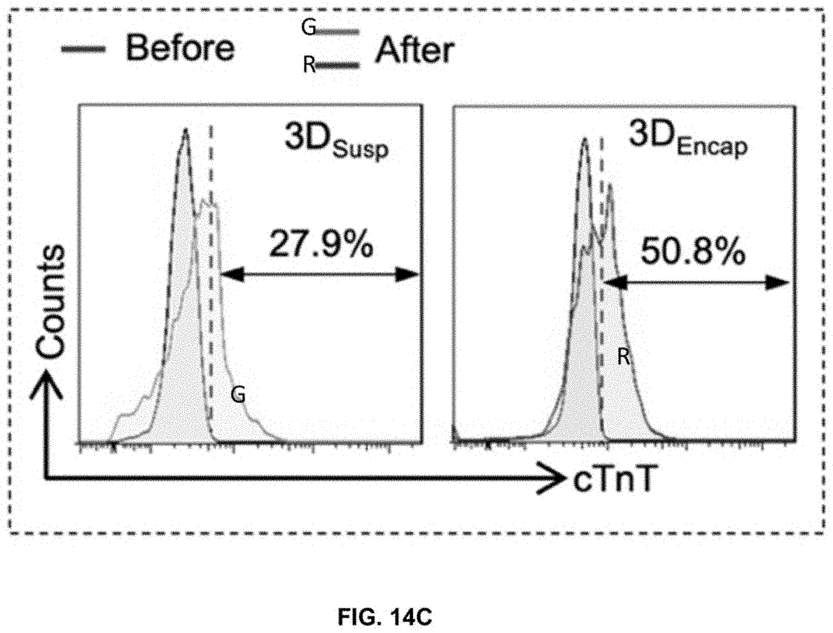

Further, a method for isolating highly pluripotent human iPSC spheroids for downstream biomedical applications is disclosed, wherein guided 3D cardiac differentiation is conducted with human iPSC spheroids from both the bioinspired 3D_{Encap} and conventional 3D_{Susp} cultures. As shown in FIG. 14A, although beating cardiac spheroids can be first observed on day 6 (after initiating differentiation) for both conditions, in embodiments the cumulative percentage (˜91%) of beating cardiac spheroids of the 3D_{Encap} group is significantly higher (˜66%) than that of the 3D_{Susp} condition on day 10. Furthermore, the expressions of the specific cardiomyocyte marker, cardiac troponin T (cTnT), of the post-differentiation spheroids obtained from both groups were examined by flow cytometry with cells from the human iPSC spheroids before differentiation as controls.

FIG. 14B-14C shows flow cytometry analysis of the cells after 10 days of guided cardiac differentiation showing significantly higher percentage of cardiac troponin T (cTnT, a protein marker specific to cardiomyocytes) positive cells in 3D_{Encap} group than that in 3D_{Susp} group (FIG. 14B). Cells from human iPSC spheroids before differentiation are used as the control (FIG. 14C). As shown in FIG. 14B, the percentage of cTnT positive cells is significantly higher for the 3D_{Encap} (50.8±4.2%) than the 3D_{Susp} (27.9±8.6%) group. Data are presented as mean±standard deviation (s.d.). Student's t-test (unpaired, two-tailed) was used to assess statistical significance. These results confirm that, in embodiments, the “Zona-like” microcapsules with HA in their core can selectively isolate the subset (˜20%) of human iPSCs with high capacity of guided differentiation (e.g., reduced differentiation resistance), to proliferate into the “ICM-like” highly pluripotent human iPSC spheroids.

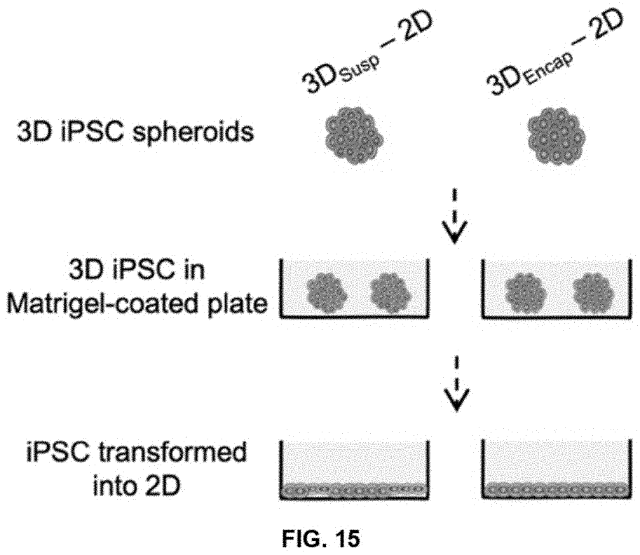

Further disclosed are methods for maintenance of stem cells at high pluripotency after transferring them back to 2D culture. In embodiments, experiments compared the expression of pluripotency proteins (NANOG, OCT4, SOX2, and SSEA-4) in the 2D-cultured cells transferred back from both the bioinspired 3D_{Encap} culture and conventional 3D_{Susp} culture, with respect to the human iPSCs without experiencing any 3D culture (2D_{Ctrl}). To do so, in embodiments human iPSC spheroids are placed onto Matrigel-coated plates to let the cells attach (FIG. 15). The cells are highly viable, and all the cells are able to attach and spread into 2D monolayers within 24 h, although the cell spreading is slightly slower for the conventional 3D_{Susp} culture than the bioinspired 3D_{Encap} culture. This observation is likely related to the large size of some of the spheroids from the conventional 3D_{Susp} culture (FIG. 16A-16C). FIG. 15 shows a schematic illustration showing conversion of 3D human iPSC spheroids from 3D_{Susp} and 3D_{Encap} cultures to 2D (3D_{Susp}-2D and 3D_{Encap}-2D) human iPSCs on Matrigel-coated plates. The human iPSCs attach on the substrate within 24 h for both conditions. FIG. 16A-16C show the phase contrast images corresponding to FIG. 15 including timepoints at time zero (FIG. 16A), 8 hours (FIG. 16B), and 24 hours (FIG. 16C).

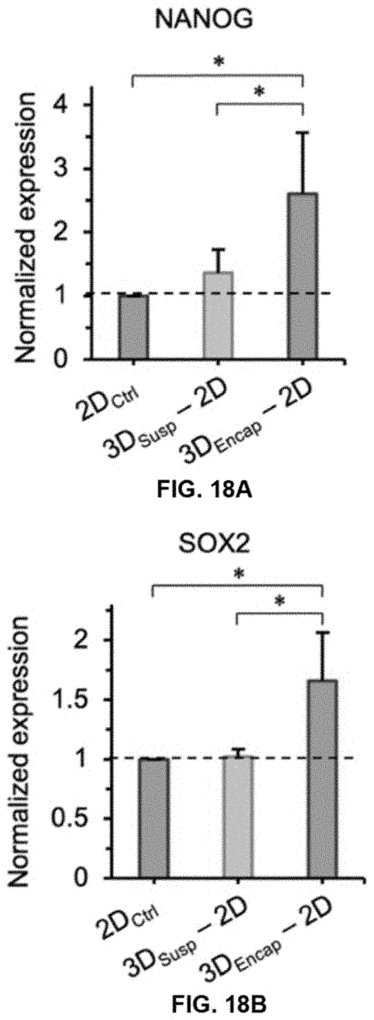

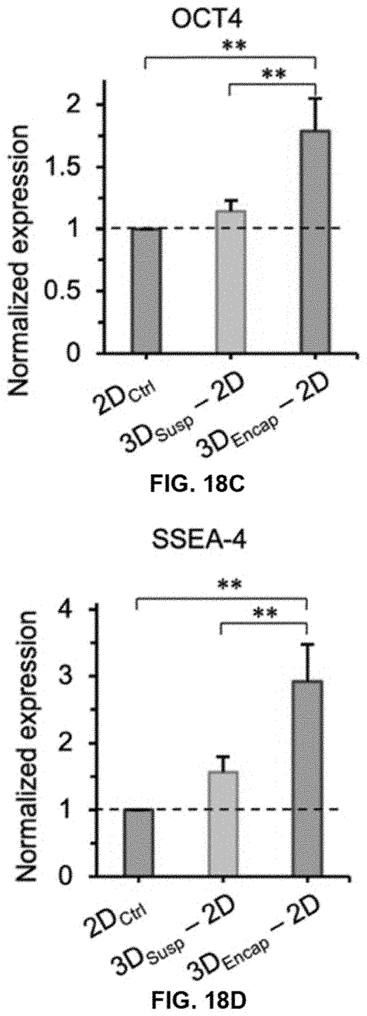

In embodiments, quantitative analyses with flow cytometry may be used to quantitate the difference in the pluripotency protein expression between the attached human iPSCs derived from the 3D_{Susp} spheroids and the 2D_{Ctrl} culture (for example, see FIGS. 17A-17D and FIG. 18A-18D). This further supports the mechanism illustrated in FIG. 9A-9C, which conveys that conventional 3D_{Susp} culture is associated with uncontrolled merging/fusion of human iPSC clusters/spheroids that are heterogeneous in pluripotency, and the human iPSCs in the resultant spheroids retain their original heterogeneity in 2D culture. In contrast, the expression of all the pluripotency protein markers is significantly upregulated in the attached human iPSCs derived from the human iPSC spheroids obtained with the bioinspired 3D_{Encap} culture, compared with not only the 2D_{Ctrl} human iPSCs but also the attached human iPSCs derived from the human iPSC spheroids obtained with the conventional 3D_{Susp} culture (See FIG. 17A-17D and FIG. 18A-18D). This shows that early embryo-like microcapsules with a nanoliter HA-rich core and “Zona-like” shell can provide a biomimetic and miniaturized 3D microenvironment together with proliferation-only culture, to selectively isolate the highly pluripotent subset (˜20%) of human iPSCs that can proliferate into the “ICM-like” highly pluripotent human iPSC spheroids.

FIG. 17A-17D depict flow cytometry peaks for expression of pluripotency markers NANOG (FIG. 17A), OCT4 (FIG. 17B), SOX2 (FIG. 17C), and SSEA-4 (FIG. 17D) in the 2D attached 3D_{Susp}-2D and 3D_{Encap}-2D human iPSCs converted from the 3D_{Susp} and 3D_{Encap} human iPSC spheroids, respectively. Data are presented as mean±standard deviation (s.d.). One-way analysis of variance (ANOVA) with Tukey's post hoc analysis was used to assess statistical significance, *p<0.05, **p<0.01. FIG. 18A-18D is the quantitative analysis corresponding to FIG. 17A-17D for expression of pluripotency markers NANOG (FIG. 18A), SOX2 (FIG. 18B), OCT4 (FIG. 18C), and SSEA-4 (FIG. 18D) in the 2D attached 3D_{Susp}-2D and 3D_{Encap}-2D human iPSCs converted from the 3D_{Susp} and 3D_{Encap} human iPSC spheroids, respectively. Data are presented as mean±standard deviation (s.d.). One-way analysis of variance (ANOVA) with Tukey's post hoc analysis was used to assess statistical significance, *p<0.05, **p<0.01.

In some embodiments, the encapsulated stem cells are treated with one or more differentiation agents to produce an encapsulated pre-differentiated stem cell or stem cell cluster (e.g., a pre-differentiated human iPSC, iPSC cluster, CSC, or CSC cluster). Pre-differentiation helps to prevent teratoma formation. For example, the encapsulated stem cells can be treated with one or more of BMP-4 and bFGF to direct the stem cells toward the mesodermal-early cardiac lineage before transplantation. Embryonic stem cells can be treated with EGF and bFGF to induce the differentiation of neural progenitor cells before implantation. In one embodiment, the encapsulated human iPSC cluster is treated with one or more differentiation agents to produce an encapsulated pre-differentiated iPSC cluster. Implanted progenitor cells can be differentiated to astrocytes, oligodendrocytes, and mature neurons. This can be used therapeutically for neural disorder treatments or spinal cord injuries. Mesenchymal stem cells can be cultured in specialized mediums with TGF-β to induce chondrogenic differentiation for cartilage repair. Mesenchymal stem cells can be induced with growth factors IGF-2 and BMP-9 to induce osteogenic differentiation for bone regeneration.

The encapsulated stem cells, with or without pre-differentiation, are in some embodiments released from the microcapsules prior to implantation, e.g., to mimic the physiologic process of the release of blastocyst from the zona pellucida for further differentiation. In some embodiments, the released stem cell clusters are encapsulated in a biocompatible, biodegradable core hydrogel/solution. The core hydrogel/solution can include one or more polycations and one or more polyanions. The core hydrogel/solution can be formed throughout and/or surrounding the cell clusters by sequential incubation of the cell aggregate in solutions of one or more polycations and one or more polyanions.

In embodiments, suitable polyanions and polycations can be selected in view of a number of factors, including the desired in vivo stability of the core hydrogel/solution (e.g., the desired in vivo biodegradation rate). Examples of suitable polycations include, for example, polypeptides, such as polyarginine, polylysine, polyhistidine, and polyornithine, polysaccharides, such as DEAE-dextran, chitosan, as well as synthetic polymers, such as polyallylamine or salts or quaternized derivatives thereof (e.g., polyallylamine hydrochloride), polyethyleneimine (PEI; e.g., linear PEI, branched PEI, or combinations thereof), modified derivatives of the above and mixtures thereof. Examples of suitable polyanions include, for example, polypeptides such as polyglutamic acid, polysaccharides, including alginates (e.g., sodium alginate), celluloses (e.g., cellulose sulfate), hyaluronic acid, and glycosaminoglycans such as chondroitin, proteins, such as heparin, as well as synthetic polymers, such as polystyrene sulfonate, modified derivatives of the above and mixtures thereof. In some embodiments, the polyelectrolyte complex can comprise a core hydrogel/solution, the core hydrogel/solution including one or more polyanions and one or more polycations selected from alginate, collagen, fibrin, hyaluronic acid (HA), heparin, chondroitin, poly-1-lysine, poly-1-glutamic acid, polyallylamine hydrochloride, polystyrene sulfonate, modified derivatives of the above and mixtures thereof.

For example, released cell clusters can be encapsulated in a core hydrogel/solution formed by soaking the clusters in chitosan (e.g., 0.4% w/v) and then in oxidized alginate (e.g., 0.15% w/v) (or non-oxidized if slow degradation is desired) solution, optionally repeated one or more times. In preferred embodiments, the core hydrogel/solution does not substantially increase the size of the stem cell clusters.

These clusters can also be encapsulated using the disclosed nonplanar microfluidic, coaxial electrospray methods, and the like.

In some embodiments, cell viability is improved via use of alginate, mannitol, alginate/mannitol, and/or chitosan solutions. For example, in one embodiment alginate (2%, w/v) dissolved in 0.25 M mannitol solution can be utilized to improve cell microencapsulation and viability. In this manner, cell viability is greatly improved without influencing the microcapsule morphology. In this example, cell viability is between 97% and 99% after microencapsulation.

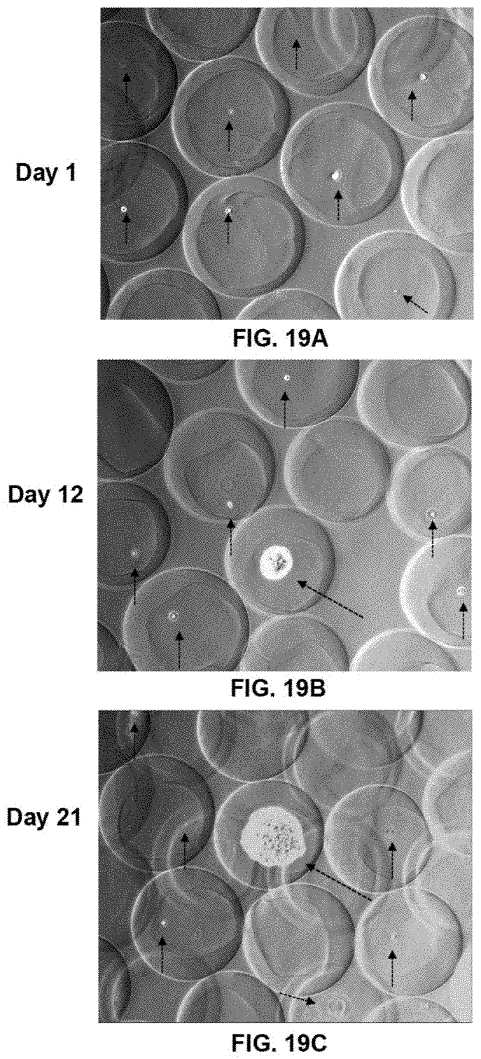

In use, the disclosed methods can also be used to isolate cancer stem cells CSCs from circulating tumor cells (CTCs). FIG. 19A-19C show phase contrast images depicting growth of circulating tumor cells (“CTCs”) into a cell colony at day 1 (FIG. 19A), day 12 (FIG. 19B), and day 21 (FIG. 19C). In embodiments, CTCs are isolated from cancer patients (e.g., breast cancer patients) and encapsulated in the core of a microcapsule. As shown by growth of the mass identified by an arrow in FIG. 19A-19C, only the cancer stem cells (CSCs) with high stemness can proliferate into a clone. The representative phase contrast images of FIG. 19A-19C show the growth of a CTC into a cell clone over 21 days under the bioinspired microencapsulation culture in DMEM. Similar to human iPSCs and multipotent ESCs, the microcapsule only allows formation of expanded cells from growth of the encapsulated cell and/or cell cluster and not fusion or merging of the cell and/or cell cluster. As a result, this approach provides for uniquely homogenous, high quality, and highly pluripotent expanded cells.

Uses of the disclosed encapsulation methods include isolating cancer stem cells from circulating tumor cells (CTCs) (e.g., mammary circulating stem cells). Methods known in the art for isolating CSCs (e.g., isolation of CSCs via markers such as CD133 and aldehyde dehydrogenase) suffer from difficulty maintaining stemness and CSC cluster homogeneity. As shown in FIG. 19A-19C, the disclosure provides both a method to isolate CSCs from CTS and a method to maintain CSC clone populations in a pluripotent and homogeneous state. These surprising results (e.g., the maintenance of a prolonged quiescent state of CSCs without loss of stemness) enables a wide range of advantages including genetic characterization of CTCs and CSCs, improved therapeutic targeting of CSCs (which are the driving force behind carcinogenesis, chemo-resistance, and radio-resistance), and new insights into the mechanisms of cancer metastasis. The application of biomimetic encapsulation technology to CTCs and CSCs further enables: (i) distinguishing CSCs from other subpopulations of cancer cells, (ii) elucidating epithelial-to-mesenchymal transition (EMT) mechanisms, and (iii) improving management of chemotherapy resistance. In embodiments, a single CSC may be encapsulated in a microcapsule, isolated, and herein proliferated at the micro scale. Similar to cardiac stem cells and other human iPSCs, CSCs can then be released from microcapsules via a PBS wash and thereafter differentiated via standard 2D methods.

Importantly, use of the herein disclosed microfluidic devices and biomimetic microencapsulation methods enables isolation of individual cells from a given mass of cells. The utility of the feature is particularly apparent in view of FIG. 19A-19C, depicting the ability of an individual cancer stem cell (CSC) to be isolated from circulating tumor cells (CTCs) and thereafter proliferated in Zona-like hydrogels. After isolation, said CSC can be proliferated in ˜4 days into a CSC clone that maintains high “cancer stemness”, a sought-after feature in the field of cancer medicine. Notably, CSCs are cultured in DMEM medium rather than StemFlex for iPSCs. In embodiments, the core hydrogel/solution of the microcapsule core may comprise DMEM Similar to human iPSCs, alginate hydrogel microcapsules washed in PBS will release the encapsulated CSC clones, thereby enabling differentiation and study of the homogenous pluripotent population via standard 2D culture methods.

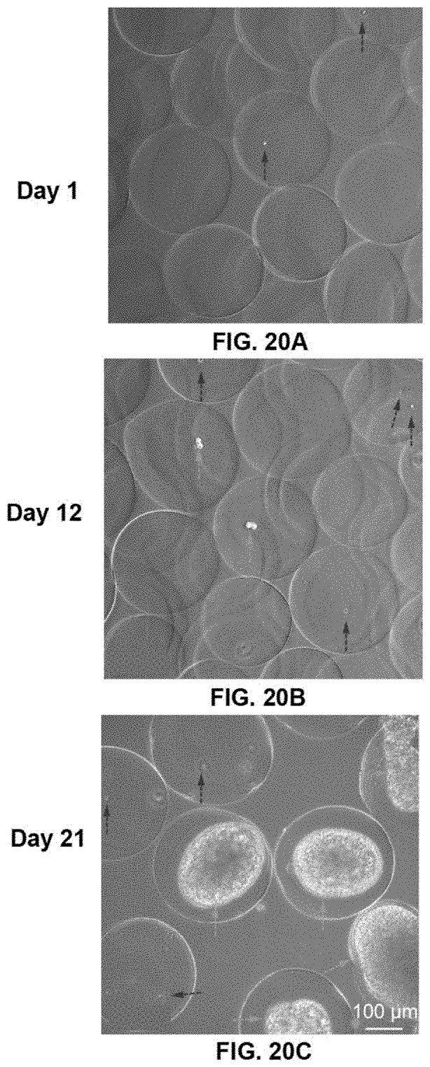

Further, the disclosed microcapsules and microculture methods may be used with glioblastoma stem cells. FIG. 20A-20C are phase contrast images showing growth of circulating glioblastoma (GBM) cells isolated from glioblastoma tumors, and the isolation of true glioblastoma stem cells (GSCs). The phase contrast images show growth at day 1 (FIG. 20A), day 12 (FIG. 20B), and day 21 (FIG. 20C). FIG. 20A-20C show phase contrast images depicting the proliferation of one single GSC into a GSC clone over 21 days in each microcapsule, while regular GBM cells that are not GSCs do not proliferate. Red arrows pointing to small cell masses indicate non-GSCs while green arrows pointing to larger spheroids indicate GSCs. As described above, this microencapsulation strategy allows for the selective isolation of GSCs with reduced differentiation resistance. GSCs with high differentiation resistance (e.g., displaying attenuated cell growth and/or limited pluripotency markers) are easily distinguishable from the rare GSCs with high viability and pluripotency (e.g., which proliferate at a faster rate and display numerous pluripotency markers) because they are sequestered in distinct microcapsules. This approach therefore represents an unparalleled means of both isolating rare GSCs and expanding GSCs without sacrificing their cancer stemness.

Exemplary circulating tumor cells, from which CSCs may be derived, include circulating solid tumor cells, such as cells derived from breast carcinomas (e.g. lobular and ductal carcinomas, such as a triple negative breast cancer), sarcomas, carcinomas of the lung (e.g., non-small cell carcinoma, large cell carcinoma, blood cancers, squamous carcinoma, and adenocarcinoma), mesothelioma of the lung, colorectal adenocarcinoma, stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, bladder carcinoma (including, for instance, transitional cell carcinoma, adenocarcinoma, and squamous carcinoma), renal cell adenocarcinoma, endometrial carcinomas (including, e.g., adenocarcinomas and mixed Mullerian tumors (carcinosarcomas)), carcinomas of the endocervix, ectocervix, and vagina (such as adenocarcinoma and squamous carcinoma of each of same), tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin), tumors of peripheral nerve, soft tissue sarcomas and sarcomas of bone and cartilage, head and neck squamous cell carcinoma, and lymphatic tumors (including B-cell and T-cell malignant lymphoma). In one example, CSCs are derived from a pancreatic adenocarcinoma CTC and are later enriched using the microencapsulation methods described herein.

Further disclosed is a kit comprising all of the elements required to formulate a microencapsulated cell including, but not limited to, a core hydrogel/solution, oil flow solution, core flow solution, shell flow solution, alginate, and microfluidic device. In one embodiment, the kit also includes gelling agents, proteins, and other cells that may be integrated into the shell and/or core. Stem cell culture media are known in the art for maintaining stem cells. In one embodiment, the culture media of the kit is StemFlex medium with Rock inhibitor. In another embodiment, the culture media comprises a mixture of DMEM and F-12 and optionally further comprises vitamins, salts, trace elements, selenium, insulin, lipids, proteins, amino acids, TGF-beta, FGF2, or mixtures thereof. Examples of typical culture media include mTeSR™1, TeSR™2, TeSR™-E8™, Essential 8™, Knockout™ D-MEM with KOSR, ReproFF™, and ReproFF2™ (ReproCell Cat #RCHEMD004, RCHEMD006, 2012). Combinations of these media formulations can be used for mesoderm, ectoderm, and endoderm induction. In one embodiment, the kit comprises a pluripotent stem cell maintenance medium. In another embodiment, the medium is TeSR™-E8™, Essential 8™, or E8.

Microfluidic Devices

Provided are microfluidic devices (e.g., a non-planar microchannel microfluidic device, and the like) configured to prepare the core-shell microcapsules (“microcapsules”) described herein.

In some embodiments, as shown in FIG. 2A-2C, a non-planar microchannel microfluidic device may be used to form a human iPSC cluster-laden core-shell microcapsule (“microcapsule”) at the flow focusing junction (FFJ) (FIG. 2A). The device can be used to isolate and culture highly pluripotent human iPSCs among a conventionally cultured heterogeneous human iPSC population. In embodiments, human iPSC clusters are suspended in a core hydrogel/solution containing 0.5% (in weight, by default) hyaluronic acid (HA) and 1% sodium alginate in saline, and the suspension is introduced into the device through a first inlet channel (FIG. 2C; “Inlet 1”; “In1”) for making the microcapsule core. In some embodiments, a solution of 2% sodium alginate in saline (for making the alginate hydrogel shell of the microcapsules) is flowed into the device through a second inlet channel (FIG. 2C; “Inlet 2”; “In2”). The mineral oil emulsified with the aqueous solution of calcium chloride (for gelling sodium alginate solution into calcium alginate hydrogel) is flowed through a third inlet channel (FIG. 2C; “Inlet 3”; “In3”). Also provided is an outlet channel where the microcapsule is extruded.

In some embodiments, alginate hydrogel is used to make the shell because it is highly biocompatible and can be easily liquified to release the encapsulated cells without damaging them^{[15d-f,19]}. Other compounds may be used to form the shell as described herein. Microencapsulation of the human iPSC clusters in the core enclosed in the semipermeable alginate shell is illustrated in FIG. 2B. FIG. 2A is a diagram showing the inlets (e.g., a first inlet channel, a second inlet channel, and a third inlet channel) and microchannel system in the non-planar microfluidic device. FIG. 2B shows a photograph of the core and shell channels while the device is in use. FIG. 2C is an embodiment of the microfluidic device including a flow focusing junction wherein a human iPSC cluster-laden core-shell microcapsule is formed.

Exemplary microchannel dimensions and flow rates used in this study are given in Table 1. Said dimensions and flow rate are optimized to achieve a high-throughput microencapsulation (˜150 microcapsules per minute) with microcapsules that are at least 357.6±18.5 μm and 268.7±27.5 μm in overall/outer and core diameter, respectively (See FIG. 4B). In some embodiments, the disclosed microcapsules can have an average diameter ranging from about 30 microns to about 1,000 microns, including about 100 microns to about 500 microns. In embodiments, the size of the core and the shell can be adjusted by varying the microchannel dimensions and/or flow rates of the fluids in the device^{[19-20]}. The overall size of the microcapsules is mainly determined by the microchannel dimensions as reported in the previous studies^{[20]}.

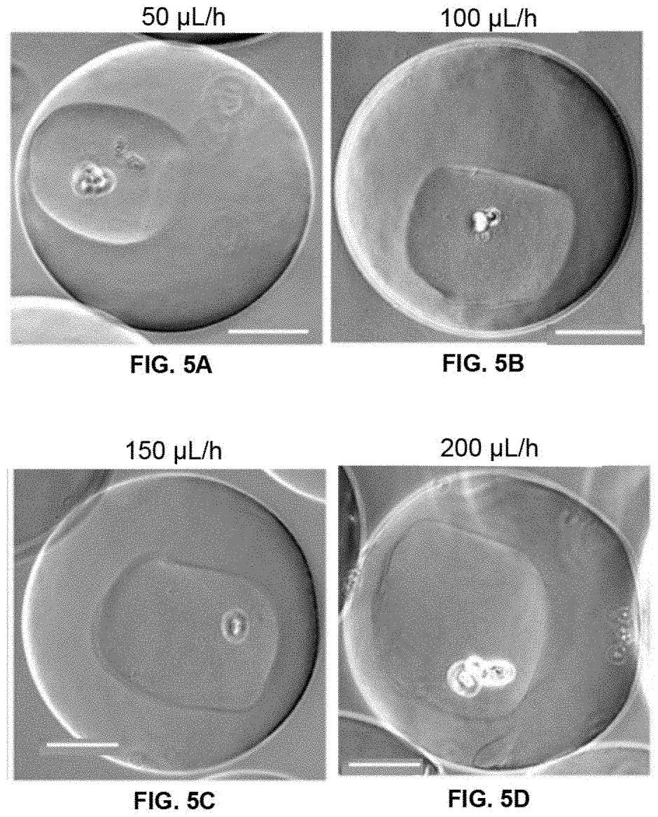

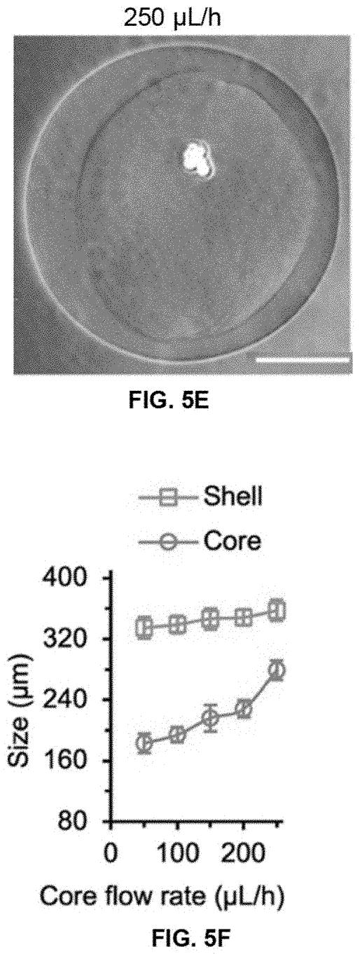

Exemplary shell and oil flow rates shown in Table 1. In embodiments, the sizes of the core are tunable by controlling the core flow rate without affecting the overall size of the microcapsules, as shown in FIG. 5A-5E and FIG. 5F. FIG. 5A-5E show representative images of the microcapsule at various flow rates including 50 microliters per hour (FIG. 5A), 100 microliters per hour (FIG. 5B), 150 microliters per hour (FIG. 5C), 200 microliters per hour (FIG. 5D), and 250 microliters per hour (FIG. 5E). In one example, the shell flow rate ranges from about 400 to about 600 microliters per hour, or about 450 to about 550 microliters per hour. Notably, in embodiments the shell is adapted to be semipermeable and/or selectively permeable to various biomolecules and small molecules. This feature is tunable by way of modifying the viscosity and/or hydrogel composition of the shell.

TABLE 1
Microchannel width (W)(microns) × depth
(D) (microns) and flow rate (microliter per hour)

| | W × D | Flow rate |
|---|---|---|
| Core | 100 × 100 | 250 |
| Shell | 150 × 150 | 500 |
| Oil | 300 × 400 | 5000 |
| Gelling | 400 × 400 | 5000 |

In embodiments, the non-planar microchannel microfluidic device includes a first inlet channel positioned between a second inlet channel and a third inlet channel, wherein the distance between the first inlet channel and the second inlet channel is more than twice as close in space than the distance between the first inlet channel and the third inlet channel. In addition, the non-planar microchannel device includes a focusing junction and an outlet channel flowing from the flow focusing junction. In some embodiments, the first inlet channel has a length of between about 1 cm and 1.4 cm. In embodiments, the length of the microfluidic device is between about 3.3 cm and 3.7 centimeters. In some embodiments, microfluidic channels have at least one cross-sectional dimension that is in the range from about 10 microns to about 750 microns, including from about 100 microns to about 750 microns, from about 1 micron to about 500 microns, from about 10 microns to about 500 microns, or from about 50 microns to about 450 microns.

The particular design of the microfluidic device, including the number and type of inlet channels with respect to the flow focusing chamber, the presence or absence of additional microfluidic components in the device, and the arrangement of the microfluidic components within the device, will be dependent upon a number of factors. These factors can include the intended composition of the microcapsules being formed by the microfluidic device, the desired microcapsule production rate, and the nature of the cells, cell clusters, and/or bioactive agents that are incorporated within the microcapsules. In the preferred embodiment, a non-planar microchannel microfluidic device is used. However, other microfluidic devices are contemplated including an electrospray apparatus configured to prepare core-shell microcapsules. Said microcapsules may be further manipulated using the methods described herein after production with an electrospray apparatus.

The dimensions of microfluidic channels may vary widely depending on the microcapsule desired (e.g., the inlet channels, shell inlet channels, crosslinker channels, outlet channel(s), etc. may be used). Said channels may be used individually and/or in combination and may be selected in view of a number of factors, including the cells and/or cellular clusters to be encapsulated, the presence or absence of cells and/or cellular clusters in the microcapsule shell, the desired microcapsule size, the desired ratio of core to shell material with the microcapsule, and combinations thereof.

The outlet channel can have any suitable length. In some instances, the outlet channel comprises an elongated and/or circuitous incubation region which is configured to increase the flow time of microcapsules within the microfluidic device. In embodiments, this can provide time for appropriate cross-linking of the microcapsule prior to the microcapsule leaving the outlet channel.

The outlet channel can also include a separator region. The separator region can be configured to transfer microcapsules formed using the microfluidic device from an organic (oil) phase to an aqueous phase. The separator region can include a separator inlet channel that converges with the outlet channel to form a separation channel. The separation channel can then diverge downstream, forming an aqueous fluid outlet channel and an organic fluid outlet channel. The separator inlet channel can be fluidly connected to a separator fluid reservoir. The aqueous fluid outlet channel and the organic fluid outlet channel can be fluidly connected to an aqueous fluid reservoir and an organic fluid reservoir respectively.

The separator region can be configured to transfer microcapsules formed using the microfluidic device from an organic (oil) phase to an aqueous phase. For example, an organic phase comprising microcapsules can flow into the separation channel from the outlet channel, and an aqueous phase can flow into the separation channel from the separator inlet channel. The separation channel can be configured to provide for laminar flow of the aqueous phase and the organic phase through the separation channel. As the microcapsules flow through the separation channel, they can be extracted from the organic phase into the aqueous phase, and flow from the separation channel into the aqueous fluid outlet channel.

The separation channel can have a length selected to provide for extraction of the microcapsules formed using the microfluidic device from the organic (oil) phase flowing through the separation channel to the aqueous phase flowing through the separation channel. An appropriate separation channel length can be selected in view of a number of factors, including the identity of the organic phase and the aqueous phase, the chemical properties of the microcapsules (e.g., microcapsule hydrophobicity/hydrophilicity), and the physical properties of the microcapsules (e.g., microcapsule elasticity). In some embodiments, the separation channel can have a length of from about 1 mm to about 15 mm (e.g., from about 1 mm to about 10 mm, or from about 3 mm to about 7 mm).

The separator region can further include a directing element. The directing element can be any suitable device component configured to apply a force to microcapsules flowing through the separation channel. For example, an organic phase comprising microcapsules can flow into the separation channel from the outlet channel, and an aqueous phase can flow into the separation channel from the separator inlet channel. The separation channel can be configured to provide for laminar flow of the aqueous phase and the organic phase through the separation channel. As the microcapsules flow through the separation channel, a directing element can apply a force to the microcapsules to direct them from the organic phase into the aqueous phase. Once in the aqueous phase, the microcapsules can flow from the separation channel into the aqueous fluid outlet channel.

For example, in some embodiments, the directing element can be a dielectrophoretic element configured to apply a non-uniform electric field to all or part of the separation channel. For example, the dielectrophoretic element can include a first dielectrophoretic channel and a second dielectrophoretic channel fluidly connected to the separation channel A potential bias can be applied across the first dielectrophoretic channel and the second dielectrophoretic channel to apply a non-uniform electric field to a portion of the separation channel. The nature of the electric field (e.g., the frequency of the electric field) can be varied as desired, for example, to direct microcapsules from the organic phase into the aqueous phase. Because the strength of the force exerted on the microcapsules is dependent on a variety of factors including the electrical properties (e.g., the composition) of the microcapsules, by varying the nature of the electric field (e.g., the frequency of the electric field), microcapsules of a particular composition (e.g., microcapsules containing encapsulated cells or cell clusters) can be selectively directed from the organic phase into the aqueous phase while microcapsules of another composition (e.g., microcapsules without encapsulated cells or cell clusters) can continue to flow through the separation channel in the organic phase.

The microfluidic device can further include one or more fluid reservoirs. In some cases, the core inlet channel is fluidly connected to a core fluid reservoir. In some embodiments, the first shell inlet channel and the second shell inlet channel are fluidly connected to a shell fluid reservoir. In certain embodiments, the first and second shell inlet channels are fluidly connected to the same shell fluid reservoir. In some embodiments, a first crosslinker inlet channel and a crosslinker shell inlet channel are fluidly connected to a crosslinker fluid reservoir. In certain embodiments, the first and second crosslinker inlet channels are fluidly connected to the same crosslinker fluid reservoir.

The microfluidic device can further include one or more additional components (e.g., pressure gauges, valves, gaskets, pressure inlets, pumps, computer-controlled solenoid valves, fluid reservoirs, and combinations thereof) to facilitate device function and microcapsule formation.

As described above, cells (e.g., stem cells, iPSCs, CSCs, and the like) are suspended in core hydrogel/solution [e.g., aqueous core hydrogel/solution containing 0.5% (in weight, by default) hyaluronic acid (HA) and 1% sodium alginate in saline], and the suspension is introduced into the device through the first inlet channel (FIG. 2C; “Inlet 1”) for making the microcapsule core. In embodiments, the solution of 2% sodium alginate in saline for making the alginate hydrogel shell of the microcapsules is then flowed into the device through the second inlet channel (FIG. 2C; “Inlet 2”; “In2”). The mineral oil emulsified with the aqueous solution of calcium chloride (for gelling sodium alginate solution into calcium alginate hydrogel) is flowed through the third inlet channel (FIG. 2C; “Inlet 3”; “In3”). In some embodiments, additional materials, such as bioactive agents (e.g., small molecule therapeutics, cells, biomolecules, nanoparticles and microparticles for drug delivery, and combinations thereof), can be incorporated into the microcapsule shell by including them in the suspension flowed through the second inlet channel. Similarly, additional materials may be added to the core by including them in the suspension that is flowed into the first inlet channel, thereby forming a heterogenous core.

In some embodiments, a crosslinking agent can be integrated into the shell, and in some embodiments may be UV activated. For example, the crosslinking agent can be a divalent metal ion, such as calcium ions, barium ions, or combinations thereof, which can react to crosslink alginate. Other suitable crosslinking agents include, for example genipin, thrombin, polycationic polymers such as polylysine, and combinations thereof. In some embodiments, crosslinking can be performed using UV irradiation and/or other high intensity wavelengths in the near visual spectrum.

The microfluidic devices described herein (e.g., a non-planar microchannel microfluidic device) can be composed of any material suitable for the flow of fluid through microfluidic channels. For example, in some embodiments, the device may be fabricated from a material that is chemically resistant to solvents. In other embodiments, the device may be fabricated with a 3D printer. In embodiments, design of the device may be augmented by artificial intelligence and/or neural networks.

In some embodiments, the device is fabricated, in whole or in part, from glass, silicon, or combinations thereof. In some embodiments, the device is fabricated, in whole or in part, from a metal and/or metal alloys (e.g., iron, titanium, aluminum, gold, platinum, chromium, molybdenum, zirconium, silver, niobium, alloys thereof, etc.). In some embodiments, the device is fabricated, in whole or in part, from a polymer and/or plastic, including, but not limited to, polyesters, polycarbonate, polyethylene terephthalate (PET) polyethylene terephthalic ester (PETE), polytetrafluoroethylene (PTFE), polymethyl methacrylate (PMMA), polydimethylsiloxane (PDMS), polyurethane, bakelite, polyester, etc. The device can also be fabricated, in whole or in part, from a ceramic (e.g., silicon nitride, silicon carbide, titania, alumina, silica, etc.).

In certain embodiments, the device is fabricated, in whole or in part, from a photocurable epoxy. In certain embodiments, the device is fabricated, in whole or in part, from polydimethylsiloxane. The device can be fabricated using a variety of suitable methods known in the art. For example, the device described herein can be formed by, for example, via lithography, etching, embossing, or molding of a polymeric surface. In general, the fabrication process may involve one or more of any of the processes described herein, and different parts of a device may be fabricated using different methods, and subsequently assembled or bonded together to form the final microfluidic device. Suitable fabrication methods can be selected in view of a number of factors, including the nature of the substrate(s) used to form the device as well as the dimensions of the microfluidic features making up the device. In some embodiments, microchannels can be directly machined into a substrate by laser machining or CNC machining. If desired, several layers thus machined can be bonded together to obtain the final device.

In embodiments, the microcapsules described herein are formed in a continuous process when flow of the component solutions is maintained. The flow rates of the component solutions can be varied to the selected characteristics of the microcapsules. FIG. 5F shows that core flow rates from 50 to 2500 microliters per hour produce microcapsules with varying core and shell sizes. In certain embodiments, the fluid flow rate through the first inlet (FIG. 2C; “Inlet 1”; “Int”) and the second inlet (FIG. 2C, “Inlet 2”; “In2”) range from about 50 microliters/hour to about 5000 microliters/hour (e.g., from about 75 microliters/hour to 175 microliters/hour, or from about 100 microliters/hour to 150 microliters/hour).

In other embodiments, the fluid flow rate through Inlet 1 (FIG. 2C; “In1”) and Inlet 2 (FIG. 2C, “In2”) range from about 80 microliters/hour to about 500 microliters/hour (e.g., from about 65 microliters/hour to about 135 microliters/hour, or from about 75 microliters/hour to about 125 microliters/hour). Suitable flow rates can be adjusted, as required, for operation of a microfluidic device having a particular architecture.

### EXAMPLES

### Example 1

Materials. Polydimethylsiloxane (PDMS) kit containing the PDMS pre-polymer, and its crosslinking agent was purchased from Dow Corning (Sylgard 184, Midland, Mich., USA). SU8 2100 and 2025 were purchased from MicroChem (Westborough, Mass., USA). Carboxymethyl cellulose sodium salt (high viscosity), sodium alginate were purchased from Sigma (St. Louis, Mo., USA) and further purified as described previously^{[24]}. Hyaluronic acid (HA, 151-300 kDa) was purchased from Acros Organics (Fair Lawn, N.J., USA). All other chemicals were purchased from Sigma unless specifically mentioned otherwise.

### Example 2

Human iPSC culture. Human iPSCs (DF19-9-11T.H) from WiCell (Madison, Wis., USA) were used for all studies in this work. For 2D culture, the human iPSCs were maintained in StemFlex medium (ThermoFisher Scientific, Waltham, Mass., USA) in Matrigel (Corning, Corning, N.Y., USA)-coated 6-well plates (1×10⁶ cells in 1.5 mL of medium per well) with medium being changed daily. They were detached and dissociated with Versene (ThermoFisher Scientific) every 3-5 days for passaging at a 1:6 ratio or prepared for 3D suspension/microencapsulation culture. For 3D suspension culture, 2×10⁶ 2D human iPSCs were collected as aforementioned and transferred to an uncoated petri dish in 10 mL of StemFlex medium with 10 μM Rock inhibitor (RI, Selleck Chemicals, Houston, Tex., USA) to form stem cell clusters (e.g., spheroids). Medium (supplemented with 10 μM RI) was changed every other day. The human iPSC spheroids were collected and dissociated into single cells for flow cytometry analyses. The human iPSC-laden microcapsules were cultured in 6-well plates (without Matrigel coating) in 2 mL StemFlex medium (supplemented with 10 μM RI) per well. Every other day, 0.5 mL of fresh medium (supplemented with 10 μM RI) was added to each well.

Preparation of Reagents. In some cases, the core of the microcapsules was composed of viscous carboxymethyl cellulose, collagen, sodium alginate, and/or hyaluronic acid solution. In one example, 10 mg/ml solution of cellulose in 0.3 M Mannitol was prepared. 3-6 mg/ml neutral collagen solution was prepared as per the manufacturer's protocol (BD Biosciences). Shell of the microcapsules was composed of 2% alginate solution prepared in 0.3 M Mannitol solution. All the solutions were buffered with 10 mM HEPES solution to maintain pH 7.2. In order to crosslink alginate in the microfluidic channel, calcium chloride was infused in mineral oil. Briefly, stable emulsion of mineral oil and calcium chloride solution (volume ratio-3:1) was prepared with the addition of 1.2% SPAN 80 as surfactant. Thereafter, water in the emulsion was evaporated using a rotary evaporator. Oil with calcium chloride solution remained stable and was used as a continuous phase in the microfluidic channel for microcapsule formation.

### Example 3

Fabrication of microfluidic devices. The non-planar PDMS-based microfluidic device was fabricated as described previously^{[19,24,27].} Briefly, a silicon master with patterned non-planar microfluidic channels was prepared by utilizing a 3-layer SU8 fabrication technique as detailed elsewhere^{[19,24]}. To fabricate PDMS-based microfluidic devices, the PDMS pre-polymer was mixed with its curing agent at a 10:1 ratio (wt/wt). Then, the mixture was plated onto the silicon master followed by baking at 75° C. for 5 h. Thereafter, two PDMS slabs with identical channel designs were plasma treated for 1 min using a Harrick PDC-32G plasma cleaner, stacked on one another, and aligned under a microscope to form an assembled non-planar microfluidic device. Finally, the devices were kept at 75° C. for at least 3 days to make the microchannel surface sufficiently hydrophobic for further experimental use.

### Example 4

Fluids for microencapsulation. The 2% alginate, 1% HA, and 1% carboxymethyl cellulose (CMC) solutions were made by dissolving the purified alginate, HA, and CMC in 0.9% saline, respectively, followed by filtering through a 0.22 μm filter (Merck Millipore, Cork, IRL). The 2% alginate solution was used as the shell fluid. The fluid in the core (1% alginate+0.5% HA, or 1% alginate+0.5% CMC) was made by mixing an equal volume of 2% alginate and 1% HA or CMC solutions. A mixture of mineral oil (15 mL), aqueous CaCl₂) solution (1.0 g/mL, 1.5 mL), and SPAN80 (93.3 μL) was sonicated for 1 min using a Branson 450 sonifier (22% power) to make the oil emulsion. For other encapsulation conditions, the fluids were prepared in the same way as that described above.

### Example 5

Microencapsulation of human iPSC clusters. The 2D cultured human iPSC were treated with Versene, gathered with gentle washing of the well bottom, and centrifuged for 45 s at 300 g. Then, the cells were gently pipetted 5 times into clusters and resuspended in the core hydrogel/solution at 3×10⁵ clusters ML⁻¹. All the solutions were injected into the microfluidic device using syringe pumps (Harvard Apparatus, Holliston, Mass., USA) to generate microcapsules. Flow rates for core, shell, and oil emulsion fluids were 250 μL h⁻¹, 500 μL h⁻¹, and 5 mL h⁻¹, respectively. The device outlet was submerged in a 50 mL tube containing 30 mL DMEM/F12 medium (ThermoFisher Scientific) to collect the microcapsules on ice. Notably, DMEM medium is used for microencapsulation of CTCs in addition to CSCs isolated from said CTCs. After collecting for 1 h, the microcapsules naturally sunk down to the bottom of the tube. After aspirating the supernatant including oil and 25 mL of medium, the remaining 5 mL of medium with microcapsules was transferred to a new 50 mL tube by pipetting. Thereafter, an equal volume (5 mL) of Calcium dichloride (CaCl₂)) (200 mM) was added to the tube to further gel alginate in the microcapsule for 45 s. The microcapsules were then rinsed using 25 mL of DMEM/F12 three times to remove residual mineral oil emulsion and subsequently transferred into a 6-well plate for culture as aforementioned. For other encapsulation conditions, the microencapsulation process was the same as that described above using indicated core flow rate (FIG. 5F).

### Example 6

Microencapsulation of mammary circulating tumor cells (CTCs) for isolating breast cancer stem cells. The isolated circulating tumor cells from breast cancer patient blood samples were gently resuspended in the core hydrogel/solution with a cell density of 200,000 cells mL-1. All the solutions were injected into the microfluidic device using syringe pumps (Harvard Apparatus, Holliston, Mass., USA) to generate microcapsules. The flow rates for core, shell, and oil emulsion fluids were 250 μL h−1, 500 μL h−1, and 5 mL h−1, respectively. The device outlet was submerged in a 50-mL tube containing 30 mL of DMEM (ThermoFisher Scientific) to collect the microcapsules on ice. After collecting for 1 h, the microcapsules naturally sunk down to the bottom of the tube. After aspirating the supernatant including oil and 25 mL of medium, the remaining 5 mL of medium with microcapsules was transferred into a new 50-mL tube by pipetting. Thereafter, an equal volume (5 mL) of calcium dichloride (CaCl2,200 mM) was added to the tube to further gel alginate in the microcapsule for 45 s. The microcapsules were then rinsed using 25 mL of DMEM three times to remove residual mineral oil emulsion and subsequently transferred into a 6-well plate for culture.

### Example 7

Microencapsulation of human glioblastoma (GBM) cells for isolating glioblastoma cancer stem cells. The suspension-cultured patient-derived GBM neurospheres were gathered and centrifuged at 100 g for 3 min. The neurospheres were then dissociated with Accutase (Sigma) for 5 min at room temperature, pipetted gently, and then centrifuged at 200 g for 3 min. The cells were resuspended in the core hydrogel/solution at 5×104 cells mL-1. All the solutions were injected into the microfluidic device using syringe pumps (Harvard Apparatus, Holliston, Mass., USA) to generate microcapsules. Flow rates for core, shell, and oil emulsion fluids were 200 μL h−1, 500 μL h−1, and 5 mL h−1, respectively. The device outlet was submerged in a 50-mL tube containing 30 mL 0.25 M mannitol solution to collect the microcapsules on ice. After collecting for 1 h, the microcapsules naturally sunk down to the bottom of the tube. After aspirating the supernatant including oil and 25 mL of medium, the remaining 5 mL of medium with microcapsules was transferred to a new 50-mL tube by pipetting. Thereafter, an equal volume (5 mL) of calcium dichloride (CaCl2) (200 mM) was added to the tube to further gel alginate in the microcapsule for 45 s. The microcapsules were then rinsed using 25 mL of 0.25 M mannitol solution twice to remove residual mineral oil emulsion. Microcapsules were further coated with 0.4% chitosan solution for 30 s, washed twice with 0.25 M mannitol, and coated with 0.2% alginate solution for 30 s, followed by two washes with 0.25 M mannitol. The microcapsules were subsequently transferred into a 6-well plate for culture in DMEM/F-12 (ThermoFisher Scientific) with 1×B27 (ThermoFisher Scientific), 20 ng/mL basic fibroblast growth factor, 20 ng/mL epidermal growth factor, and 1× antibiotic-antimycotic.

### Example 8

Flow cytometry analyses. All the experiments were performed in the same way as that described previously^{[24]}. Briefly, for flow cytometry analyses, human iPSCs without or with cardiac differentiation were dissociated into single cells using 0.25% trypsin-EDTA (ethylenediaminetetraacetic acid, ThermoFisher Scientific). Single human iPSCs were then fixed, permeabilized, and stained with antibodies (1:1,000 dilution using 1% BSA solution for both primary and secondary antibodies) as aforementioned. The cells were then analyzed using a BD Biosciences (San Jose, Calif., USA) FACSCelesta Flow cytometer. The primary antibodies for human iPSCs were NANOG (Santa Cruz Biotechnology, Dallas, Tex., USA), OCT4 (Abcam, Cambridge, UK), SOX2 (Sigma), and SSEA-4 (Cell Signaling Technology, Danvers, Mass.). The primary antibody for cardiomyocytes was cTnT (ThermoFisher Scientific). Secondary antibodies included either goat anti-mouse or goat anti-rabbit FITC-tagged antibodies (ThermoFisher Scientific).

### Example 9

Cardiac differentiation. After 4 days of culture in microcapsules, human iPSC spheroids were released from microcapsules by washing with phosphate-buffered saline (PBS) for 3 times and collected for cardiac differentiations. The PSC Cardiac Differentiation Kit was used as instructed by the manufacturer (ThermoFisher). Briefly, the cells were cultured in Cardiac Differentiation Medium A as given in the kit for 2 days. Then, the cells were cultured in Cardiac Differentiation Medium B as given in the kit for 2 days. For another 6 days, the cells were cultured in Cardiac Maintenance Medium as given in the kit. Medium changes were performed every other day. Differentiation of human iPSC spheroids from conventional 3D suspension culture was performed as aforementioned, as well.

### Example 10

Statistical analysis. Each experiment was independently repeated at least three times. All data are presented as mean±standard deviation (s.d.). Student's t-test (unpaired, two-tailed) was used to compare between two groups. One-way analysis of variance (ANOVA) with Tukey's post hoc analysis was used to compare between different groups. All statistical analyses were carried out with the Prism (version 7.0, GraphPad Software, San Diego, Calif., USA). A p value less than 0.05 was considered statistically significant.

What is claimed is:

1. A microcapsule composition for isolating and expanding cells comprising:
a. a core comprising a cell and/or cell cluster, wherein the cell and/or cell cluster is suspended in a core hydrogel/solution; and
b. a shell surrounding the core, the shell comprising a hydrogel, wherein the core hydrogel/solution and shell hydrogel are distinct in their composition/property.

2. The microcapsule composition of claim 1, wherein the core hydrogel/solution comprises hyaluronic acid (HA).

3. The microcapsule composition of claim 1, wherein the cell and/or cell cluster comprise a stem cell and/or stem cell cluster.

4. The microcapsule composition of claim 2, wherein the core has an average volume from about 0.005 nanoliters to about 500 nanoliters.

5. The microcapsule composition of claim 3, wherein the shell is semipermeable.

6. The microcapsule composition of claim 3, wherein the stem cell and/or stem cell cluster comprise a multipotent stem cell, cancer stem cells, cardiac stem cell, and/or organ-specific stem cell.

7. The microcapsule composition of claim 3, wherein the stem cell and/or stem cell cluster comprise a human induced pluripotent stem cell (iPSC) or a human iPSC cluster, and wherein the core further comprises carboxymethyl cellulose (CMC).

8. The microcapsule composition of claim 4, wherein the core has an average diameter ranging from about 20 microns to about 1,000 microns.

9. The microcapsule composition of claim 4, wherein the shell has a thickness ranging from about 10 microns to about 1,000 microns, and wherein the core further comprises collagen, laminin, and/or fibrin.

10. The microcapsule composition of claim 7, wherein the core, shell, or a combination thereof further comprise a bioactive agent.

11. A method of encapsulating one or more cells comprising: suspending a cell and/or cell clusters in at least one microcapsule, wherein the at least one microcapsule comprises a hydrogel shell, and wherein the microcapsule has an average diameter of less than about 1,000 microns.

12. The method of claim 11, wherein the suspended cell and/or cell clusters comprise iPSC and/or iPSC clusters, and wherein the iPSC and/or the iPSC clusters release autocrines and/or paracrines within the microcapsule.

13. The method of claim 12, wherein the suspended iPSC clusters comprise human iPSC clusters, and wherein the human iPSC clusters proliferate into highly pluripotent human iPSC spheroids within the at least one microcapsule, wherein the highly pluripotent human iPSC spheroids have a radius of less than about 500 microns.

14. The method of claim 12, wherein the suspended iPSC clusters are treated with one or more differentiation agents to stimulate the growth, survival, pluripotency, and/or differentiation of the iPSC clusters encapsulated in the microcapsules.

15. The method of claim 13, further comprising washing the at least one microcapsule, thereby releasing the highly pluripotent human iPSC spheroids from the microcapsule.

16. The method of claim 11, wherein the cells comprise circulating cancer cells (CTCs); wherein at least one cancer stem cell (CSC) with high stemness is isolated from the CTCs; wherein the at least one CSC proliferates into a CSC clone when cultured in DMEM or CSC medium.

17. The method of claim 16, wherein the at least one microcapsule is washed in phosphate-buffered saline (PBS), sodium citrate, EDTA, or another solution for ion chelation.

18. A method for isolating and expanding human iPSC clusters comprising: encapsulating human iPSC clusters in a core, wherein the core has a volume ranging from about 0.005 to 500 nanoliters; wherein the core is enclosed in a shell, wherein the shell comprises a hydrogel and/or other bioactive agents including cells/tissues.

19. The method of claim 18, wherein the human iPSC clusters proliferate into pluripotent iPSC spheroids inside a microcapsule after 3 to 5 days of culture.

20. The method of claim 19, wherein application of isotonic solution to the microcapsule liquefies the hydrogel, thereby releasing the pluripotent iPSC spheroids from the microcapsules.

21. The method of claim 20, wherein the iPSC spheroids are cultured via conventional 2D methods after release from the microcapsules.
